(19) United States
(12) Patent Application Publication
(10) Pub. No.: US 2016/0040218 A1
(43) Pub. Date: Feb. 11, 2016

(54) Selective Purification of RNA and RNA-Bound Molecular Complexes

(71) Applicants: THE BROAD INSTITUTE, INC., Cambridge, MA (US); MASSACHUSETTS INSTITUTE OF TECHNOLOGY, Cambridge, MA (US)

(72) Inventors: Mitchell GUTTMAN, West Hollywood, CA (US); Jesse ENGREITZ, Cambridge, MA (US); Eric S. LANDER, Cambridge, MA (US)

(21) Appl. No.: 14/774,635

(22) PCT Filed: Mar. 14, 2014
(86) PCT No.: PCT/US14/27298

§ 371 Date: Sep. 10, 2015

Related U.S. Application Data
(60) Provisional application No. 61/789,069, filed on Mar. 15, 2013.

(51) Int. Cl. C12Q 1/68

(52) U.S. Cl. CPC C12Q 1/6816

(57) ABSTRACT

Disclosed are methods for isolating a target nucleic molecule of interest from a sample. The methods include contacting a sample comprising a target nucleic molecule of interest with at least one single stranded nucleic acid targeting probe comprising a nucleic acid sequence between about 30 nucleotide and the length of the target nucleic acid length that hybridizes to the target nucleic molecule of interest under highly stringent hybridization conditions. The probe comprises a capture moiety covalently linked to one or more nucleotide bases in the probe. The targeting probes can be captured with a specific binding agent that specifically binds the capture moiety, thereby isolating the target nucleic molecule of interest. In some embodiments, the sample is further contacted with a crosslinking agent before contacting the sample with a targeting probe. Thus, complexes formed with the target nucleic acid can also be isolated.

### CROSS REFERENCE TO RELATED APPLICATION

This application claims the priority benefit of the earlier filing date of U.S. Provisional Application No. 61/784,069, filed Mar. 14, 2013, which is hereby specifically incorporated herein by reference in its entirety.

### STATEMENT OF GOVERNMENT INTEREST

This invention was made with government support under grant number 5P50HG006193 awarded by the National Institutes of Health. The government has certain rights in the invention.

### FIELD OF THE DISCLOSURE

This disclosure concerns methods for the purification and/or separation of biological macromolecules, such as RNA.

### BACKGROUND

A major goal in modern biology is defining the interactions between different biological actors in vivo. Over the past few decades, major advances have been made in developing methods to identify the molecular interactions with any given protein. These include methods to identify interacting proteins using co-immunoprecipitation followed by Mass-spectrometry analysis. Methods to identify interacting DNA regions using chromatin immuneprecipitation (ChIP) followed by DNA sequencing, and methods to identify interacting RNAs using RNA immunoprecipitation (RIP) or crosslinking and immunoprecipitation (CLIP). These methods have revolutionized many areas of biology and led to great advances in our understanding of transcriptional regulation, chromatin biology and epigenetics, and RNA processing and splicing.

Yet, despite this progress in defining the interactions mediated by a specific protein, methods to define the interactions between other molecular components, such as RNA molecules, remain largely unexplored. Over the last decade, it has become clear that there are many classes of functional RNA molecules, including non-coding RNA (ncRNA). Understanding the function and molecular interactions of ncRNAs has lagged compared to our understanding of protein regulators primarily due to the limitations of tools to study ncRNAs in vivo. Therefore, the need exists for new and innovative methods of determining these interactions. This disclosure meets that need.

### SUMMARY OF THE DISCLOSURE

Disclosed are methods for isolating a target nucleic molecule of interest from a sample. The methods include: contacting a sample comprising a target nucleic molecule of interest with at least one single stranded nucleic acid targeting probe, that targeting probe comprising a nucleic acid sequence between about 30 nucleotide bases in length to the length of the target nucleic acid that hybridizes to the target nucleic molecule of interest under highly stringent hybridization conditions. The probe includes a capture moiety covalently linked to one or more nucleotide bases in the targeting probe and facilitates capture of the targeting probe. The targeting probes are captured via the capture moiety, thereby isolating the target nucleic molecule of interest.

In some embodiments, the sample is further contacted with a protein-nucleic acid crosslinking agent, a nucleic acid-nucleic acid crosslinking agent, a protein-protein crosslinking agent or any combination thereof before contacting the sample with a targeting probe. In some embodiments, the captured probes are washed at a temperature of up to about 55° C., for example to remove molecules that non-specifically bind to the targeting probe, the target nucleic molecule of interest, proteins and/or nucleic acids that are crosslinked to the target nucleic molecule of interest, or any combination thereof. In some embodiments, the captured targeting probes are eluted. In some embodiments, the identity of the proteins and/or nucleic acids crosslinked to the target nucleic acid is determined.

Also disclosed are probes for use in the methods disclosed herein and kits containing such probes.

The foregoing and other features of this disclosure will become more apparent from the following detailed description of several embodiments, which proceeds with reference to the accompanying figures.

### BRIEF DESCRIPTION OF THE FIGURES

FIG. 1 is a schematic showing an overview of an exemplary RAP procedure. Cells are crosslinked and RNA captured using a set of antisense RNA oligos that are linked to capture moieties. The associated complexes can be detected by sequencing and/or Mass-Spectrometry.

FIG. 2 is a schematic showing an overview of an exemplary RAP procedure. Cells are crosslinked and RNA captured using a set of antisense biotinylated RNA oligos.

FIG. 3 is a schematic of exemplary probes targeting many genes, which can be synthesized in pools, for example using microarrays. Unique primers allow for amplification of specific subsets of probes from the pool.

FIG. 4 is a schematic showing probes designed with multiple overlapping primers on either end, allowing for amplification of many overlapping subsets while avoiding an increase in the number of total number of oligos synthesized.

FIGS. 5A-5D is a set of bar graphs and a digital image showing RAP in crosslinked cells captures molecular partners of target RNAs. (FIG. 5A) RAP pulldown for Xist cells produces high enrichments and yields for target RNA. (FIG. 5B) Targeting Xist RNA, which coats the inactive X chromosome in mammalian females, enriches chromosome X DNA. (FIG. 5C) Targeting 18S rRNA enriches other ribosomal RNA. (FIG. 5D) Pulldown for 28S rRNA enriches the ribosomal protein Rp17.

FIG. 6 is a graph showing 18S enrichment from purified RNA samples is tunable by adjusting the hybridization stringency, which is controlled by factors including temperature, salt concentration, and presence of denaturing reagents (urea, formamide, or guanidinium thiocyanate).

FIG. 7 is a schematic and a chart showing the information provided by differential crosslinking methods to assemble lincRNA complexes

FIGS. 8A-8C is a set of schematics and bar graphs showing the different outcomes for different classes of crosslinkers. (FIG. 8A) Distinguishing localization models using direct nucleic acid crosslinking and protein mediated crosslinking (FIG. 8B) AMT provides high efficiency nucleic acid crosslinking (black) which can be efficiently reversed (grey). (FIG. 8C) RAP efficiently captures RNA in ATM crosslinked extracts (black) compared to non-crosslinked (grey).

FIG. 9 is a schematic diagram showing the non-specific interactions and specific interactions occurring in a sample.

FIG. 10 is a schematic showing how psoralens crosslink nucleic acids.

FIG. 11 is set of schematics showing that interactions between two different RNAs can be categorized as direct or indirect.

FIG. 12A-12C is a set of schematics and graphs showing U1 binds 5′ splice sites and cryptic motifs through nascent transcripts. FIG. 12A, Schematic diagram of U1 RAP-RNA^{[AMT]}. AMT (4′-aminomethyltrioxalen) forms covalent crosslinks (orange) between opposing uridine bases in the U1 snRNA (red) and target RNA (gray). U1 is captured using biotinylated probes (blue), and purified RNAs are sequenced through 3′ adapter ligation and reverse transcription so that complementary DNA ends (cDNAs, purple) correspond to crosslinking sites. FIG. 12B, RNA-sequencing second-strand read counts averaged over all 5′ splice sites. To visualize crosslink sites, each read contributes a count in the single base at the 5′ end of the read. U1 RAP-RNA was performed in cells crosslinked with AMT (+AMT, red) or mock-crosslinked with DMSO (−AMT, blue). Crosslinked input RNA (black) is show for comparison. Pileup of reads in U1+AMT but not in U1−AMT or Input shows that U1 RAP-RNA^{[AMT]} occurs 8 bases downstream of the 5′ splice site, precisely where an AMT crosslink would occur in a canonical U1-pre-mRNA interaction. FIG. 12C, Enrichment (U1 RAP-RNA versus input) for every possible 8-mer sequence. 8-mer counts include the second strand read and the 30 bases upstream of the read. Colored dots represent 8-mers that are significantly enriched in U1 RAP versus input (P<0.001 after Bonferroni correction). The most enriched 8-mer (red label) perfectly matches the consensus 5′ splice site motif. Red, orange, and yellow dots correspond to nmers that have a Levenshtein edit distance from the 8-mer consensus motif of 1, 2, or >2, respectively.

### DETAILED DESCRIPTION OF SEVERAL EMBODIMENTS

### I. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of specific terms are provided:

Amplification: To increase the number of copies of a nucleic acid molecule. The resulting amplification products are called “amplicons.” Amplification of a nucleic acid molecule (such as a DNA or RNA molecule encoding targeting probe) refers to use of a technique that increases the number of copies of a nucleic acid molecule (including fragments).

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Antibody: A polypeptide ligand comprising at least a light chain or heavy chain immunoglobulin variable region which specifically recognizes and binds an epitope of an antigen, such as a protein crosslinked (either directly or indirectly) to a target nucleic acid of interest, or a fragment thereof. Antibodies can include a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

Binding or stable binding (of an oligonucleotide): An oligonucleotide, such as a targeting probe, binds or stably binds to a target nucleic acid if a sufficient amount of the oligonucleotide forms base pairs or is hybridized to its target nucleic acid. Binding can be detected by either physical or functional properties. In some embodiments, a targeting probe stably binds a target nucleic acid under strongly denaturing conditions.

Binding site: A region on a protein, DNA, or RNA to which other molecules stably bind. In one example, a binding site is the site on a RNA molecule, such as a lincRNA, that proteins and/or nucleic acids bind.

Biotin-16-UTP: A biologically active analog of uridine-5′-triphosphate that is readily incorporated into RNA during an in vitro transcription reaction by RNA polymerases such as T7, T3, or SP6 RNA Polymerases. In some examples, biotin-16-UTP is incorporated into an RNA targeting probe during reverse transcription from a probe DNA template, for example during in vitro transcription with an RNA Polymerase, such as T7, T3, or SP6 RNA Polymerase.

Capture moieties: Molecules or other substances that when attached to a targeting probe allow for the capture of the targeting probe through interactions of the capture moiety and something that the capture moiety binds to, such as a particular surface and/or molecule, such as a specific binding molecule that is capable of specifically binding to the capture moiety.

Contacting: Placement in direct physical association, including both in solid or liquid form, for example contacting a sample with a targeting probe and/or a crosslinking agent.

Conditions sufficient to detect: Any environment that permits the desired activity, for example, that permits an antibody to bind an antigen, such as a protein that has been or is crosslinked to a target nucleic acid sequence.

Control: A reference standard. A control can be a known value or range of values indicative of basal levels or amounts or present in a tissue or a cell or populations thereof (such as a normal non-cancerous cell). A control can also be a cellular or tissue control, for example a tissue from a non-diseased state and/or exposed to different environmental conditions. A difference between a test sample and a control can be an increase or conversely a decrease. The difference can be a qualitative difference or a quantitative difference, for example a statistically significant difference.

Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between a target nucleic acid and a protein and/or nucleic acid that has been crosslinked to the target nucleic acid by chemical means.

Crosslinking agent: A chemical agent or even light, that facilitates the attachment of one molecule to another molecule. Crosslinking agents can be protein-nucleic acid crosslinking agents, nucleic acid-nucleic acid crosslinking agents, and protein-protein crosslinking agents. Examples of such agents are known in the art. In some embodiments, a crosslinking agent is a reversible crosslinking agent. In some embodiments, a crosslinking agent is a non-reversible crosslinking agent.

Detect: To determine if an agent (such as a signal or particular nucleic acid probe, or protein) is present or absent. In some examples, this can further include quantification in a sample, or a fraction of a sample, such as a particular cell or cells within a tissue.

Detectable label: A compound or composition that is conjugated directly or indirectly to another molecule to facilitate detection of that molecule. Specific, non-limiting examples of labels include fluorescent tags, enzymatic linkages, and radioactive isotopes. In some examples, a label is attached to an antibody or nucleic acid to facilitate detection of the molecule antibody or nucleic acid specifically binds.

DNA sequencing: The process of determining the nucleotide order of a given DNA molecule. Generally, the sequencing can be performed using automated Sanger sequencing (AB13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®).

In some embodiments, DNA sequencing is performed using a chain termination method developed by Frederick Sanger, and thus termed “Sanger based sequencing” or “SBS.” This technique uses sequence-specific termination of a DNA synthesis reaction using modified nucleotide substrates. Extension is initiated at a specific site on the template DNA by using a short oligonucleotide primer complementary to the template at that region. The oligonucleotide primer is extended using DNA polymerase in the presence of the four deoxynucleotide bases (DNA building blocks), along with a low concentration of a chain terminating nucleotide (most commonly a di-deoxynucleotide). Limited incorporation of the chain terminating nucleotide by the DNA polymerase results in a series of related DNA fragments that are terminated only at positions where that particular nucleotide is present. The fragments are then size-separated by electrophoresis a polyacrylamide gel, or in a narrow glass tube (capillary) filled with a viscous polymer. An alternative to using a labeled primer is to use labeled terminators instead; this method is commonly called “dye terminator sequencing.”

“Pyrosequencing” is an array based method, which has been commercialized by 454 Life Sciences. In some embodiments of the array-based methods, single-stranded DNA is annealed to beads and amplified via EmPCR®. These DNA-bound beads are then placed into wells on a fiber-optic chip along with enzymes that produce light in the presence of ATP. When free nucleotides are washed over this chip, light is produced as the PCR amplification occurs and ATP is generated when nucleotides join with their complementary base pairs. Addition of one (or more) nucleotide(s) results in a reaction that generates a light signal that is recorded, such as by the charge coupled device (CCD) camera, within the instrument. The signal strength is proportional to the number of nucleotides, for example, homopolymer stretches, incorporated in a single nucleotide flow.

High throughput technique: Through a combination of robotics, data processing and control software, liquid handling devices, and detectors, high throughput techniques allows the rapid screening of potential reagents, conditions, or targets in a short period of time, for example in less than 24, less than 12, less than 6 hours, or even less than 1 hour. Through this process, one can rapidly identify active compounds, antibodies, or genes affecting a particular binding event.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when there is a sufficient degree of complementarity to avoid non-specific binding of the oligonucleotide or analog to non-target sequences under conditions where specific binding is desired. Such binding is referred to as specific hybridization. In some embodiments, a targeting probe is capable of specifically hybridizing to a target nucleic acid molecule under highly stringent conditions.

Isolated: An “isolated” biological component (such as a protein, a nucleic acid probe, such as the probes and target nucleic acids described herein) has been substantially separated or purified away from other biological components in the cell of the organism in which the component naturally occurs, for example, extra-chromatin DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

Mass spectrometry: A method wherein a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample can be subjected to one or more dimensions of chromatographic separation, for example, one or more dimensions of liquid or size exclusion chromatography.

Nucleic acid (molecule or sequence): A deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

In some examples, nucleotides include those nucleotides containing modified bases, modified sugar moieties, and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl)uracil, 2,6-diaminopurine and biotinylated analogs, amongst others. Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In some examples, nucleotides do not include those nucleotides containing modified bases, modified sugar moieties, and modified phosphate backbones

Targeting Probe: A probe, such as a targeting probe, includes an isolated nucleic acid capable of hybridizing to a target nucleic acid under highly denaturing conditions and includes a detectable label, such as biotin, attached to a nucleic acid molecule.

Target nucleic acid molecule of interest: Any nucleic acid present or thought to be present in a sample about which information would like to be obtained, such as its presence or absence and/or the molecules it interacts with, such as nucleic acids and proteins, either directly or indirectly. In some embodiments, a target nucleic acid of interest is an RNA, such as a lincRNA. In some embodiments, a target nucleic acid of interest is a DNA.

Sample: A sample, such as a biological sample, that includes biological materials (such as nucleic acid and proteins, for example double-stranded nucleic acid binding proteins) obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including without limitation, single celled organisms, such as bacteria, yeast, protozoans, and amebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue or organ.

Specific Binding Agent: An agent that binds substantially or preferentially only to a defined target such as a protein, enzyme, polysaccharide, oligonucleotide, DNA, RNA, recombinant vector or a small molecule. In an example, a “capture moiety specific binding agent” is capable of binding to a capture moiety that is covalently linked to a targeting probe.

A nucleic acid-specific binding agent binds substantially only to the defined nucleic acid, such as RNA, or to a specific region within the nucleic acid. In some embodiments a specific binding agent is a targeting probe, that specifically binds to a target nucleic acid of interest.

A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Test agent: Any agent that that is tested for its effects, for example its effects on a cell and/or interaction profile of a target nucleic acid of interest. In some embodiments, a test agent is a chemical compound, such as a chemotherapeutic agent, antibiotic, or even an agent with unknown biological properties.

Tissue: A plurality of functionally related cells. A tissue can be a suspension, a semi-solid, or solid. Tissue includes cells collected from a subject such as blood, cervix, uterus, lymph nodes breast, skin, and other organs.

Under conditions that permit binding: A phrase used to describe any environment that permits the desired activity, for example conditions under which two or more molecules, such as nucleic acid molecules and/or protein molecules, can bind. In some embodiments, conditions that permit binding are highly denaturing conditions.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting

### II. Description of Several Embodiments

A. Introduction

Disclosed herein is a method for specific capture of nucleic complexes, such as RNA complexes in vivo. The disclosed method, termed RNA Arrayed-hybrid Purification (RAP), allows the specific and sensitive purification of any given RNA molecule and its associated complexes. In specific applications, the RAP method has been designed to capture target RNAs that are crosslinked to other biological molecules, for example molecules that specifically interact with the target nucleic acid, such as target RNA. In some applications, the method is used to deplete an RNA from a sample, for example an RNA with high abundance, such a ribosomal RNA.

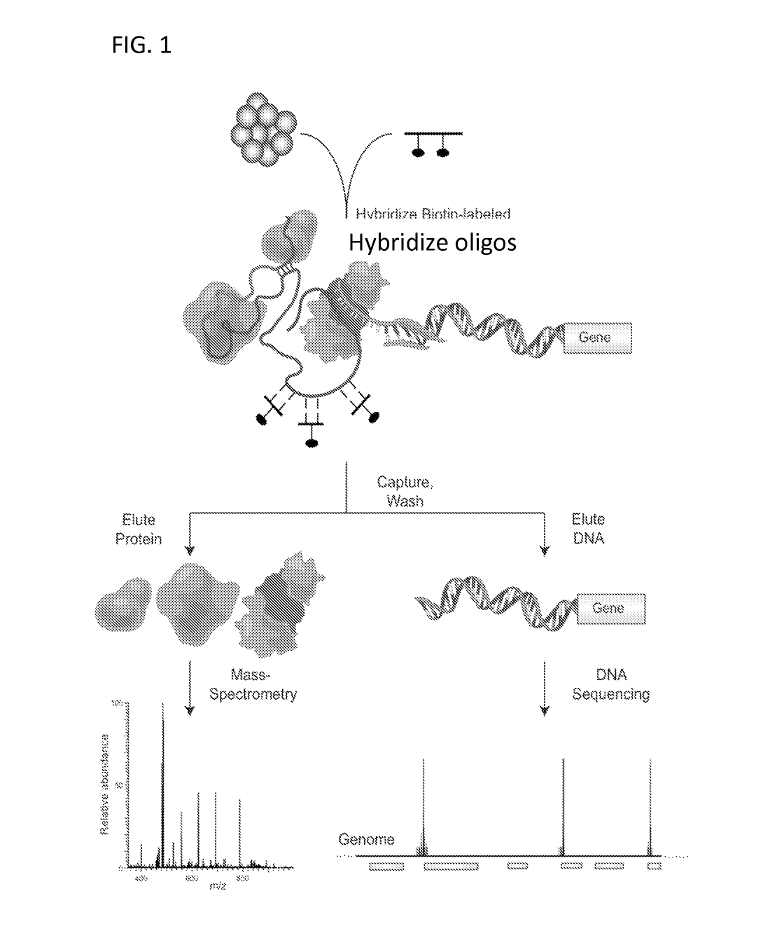

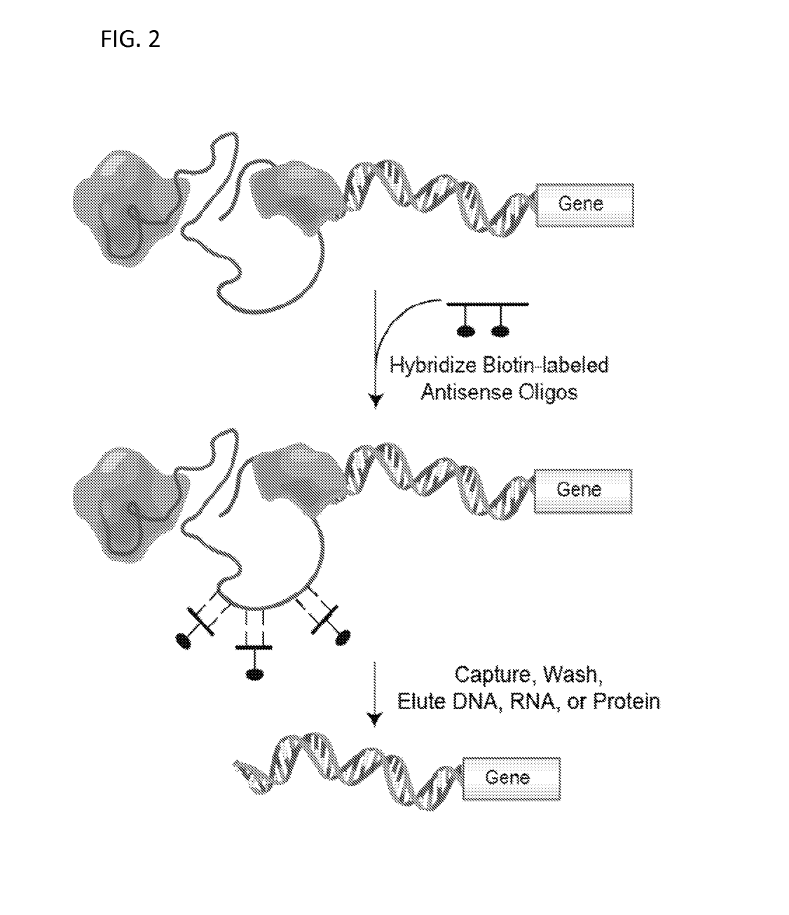

With reference to FIG. 1, and the analysis of proteins and/or nucleic acid that form complexes with a target nucleic acid, cells are treated with crosslinker (such as a protein-protein crosslinker, a protein-nucleic acid crosslinker, a nucleic acid-nucleic acid crosslinker or any combination) and the cells are lysed prior to contact with targeting probes (oligos) that includes a moiety, such as one or more moieties, that can be captured, for example by another molecule, such as a specific binding agent, or a surface the targeting agent binds to. In the example shown in FIG. 2, the capture moiety is shown as biotin and the capture moiety specific binding agent is shown as streptavidin, however, as discussed below a variety of capture moieties and specific binding agents can be used, as well as surfaces that bind capture moieties. The targeting probes are designed to a specific target nucleic acid of interest. The targeting probes bind to the target nucleic acid and are then pulled out of solution via capture of the capture moiety by some means (for example, streptavidin if the label is biotin). Non-crosslinked molecules are then washed off. The molecules are then bound to the probes and/or target nucleic acid and these molecules are available for further analysis, such as Mass spectrometry and/or sequencing.

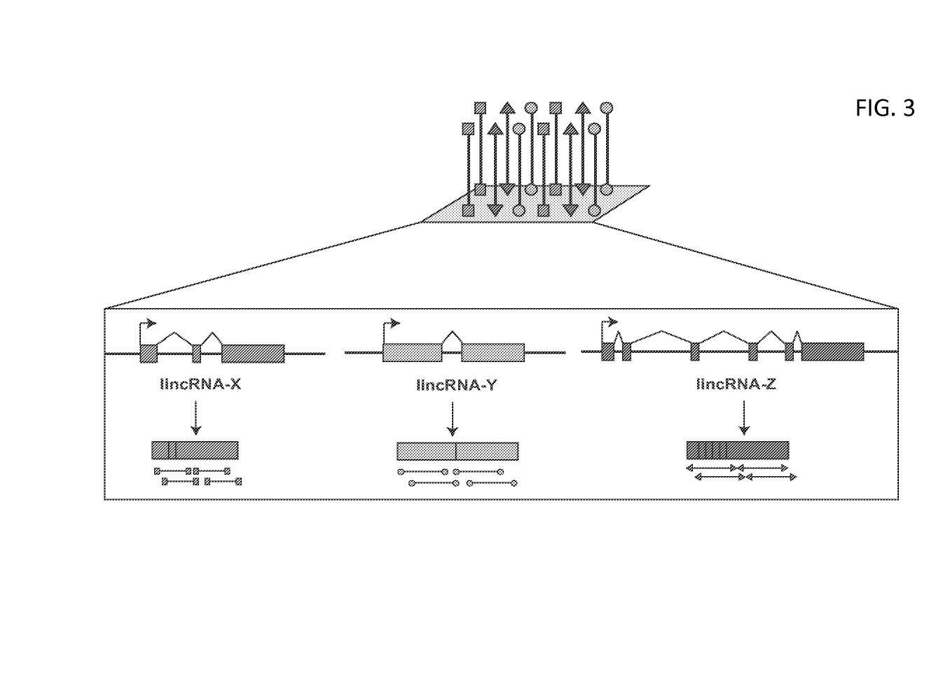

In some embodiments, the disclosed method uses long RNA probes, such as antisense RNA probes, which can be tiled across the endogenous RNA sequence to specifically and efficiently capture a given RNA. In some embodiments, these hybridized molecules are purified using specific binding agents that specifically bind these long RNA probes via capture moieties, which can be attached to a solid support, such as magnetic beads. The use of pairs of capture moieties and specific binding agents allows for the specific capture of a target RNA that is bound to the targeting probe through nucleic acid hybridization. A unique feature of the disclosed method is the use of long targeting probes, which are antisense to the target nucleic acid. The long length of the antisense probes allows the use of highly stringent hybridization conditions during the binding of the targeting probe and the target nucleic acid which results in (i) denaturation of RNA secondary structure for improved hybridization, and (ii) the elimination of non-specific molecular interactions, a well-known problem for RNA-Protein complexes. In some embodiments, the long probes are generated in pools on microarrays and designed so that subsets of probes can be amplified with specific primers. With reference to FIG. 3, the targeting probes can be made from pools of oligos that span the nucleic acid of interest, such as a lincRNA or any other RNA.

As disclosed herein, that methods provides enrichments generally exceeding ˜100-fold for target RNAs while simultaneously capturing high yields of the endogenous RNA. In a specific example, the method enables efficient capture of endogenous ncRNAs in the presence of in vivo crosslinking methods which include direct nucleic acid crosslinkers, protein crosslinkers, and protein-nucleic acid crosslinkers as well as combinations of crosslinkers. Moreover, in the presence of in vivo crosslinking the disclosed methods can specifically recover known or unknownf complexes that associate with a target RNA, including proteins, DNA loci, and other RNAs. As the method achieves high yields of target RNAs, in addition to high enrichments, this method can also be used for RNA depletion, for example to remove non-desired RNAs that may interfere with sequencing library preparation, the amplification of less abundant RNA targets, or pooled amplification of full-length RNA.

The disclosed method, can be used in conjunction with western blots and Mass-Spectrometry for detecting interacting proteins, coupled to DNA sequencing for detecting interacting genomic DNA regions, and coupled to RNA sequencing for detecting interacting RNAs.

B. RNA Arrayed-Hybrid Purification

Disclosed are methods for isolating a target nucleic molecule of interest from a sample. The disclosed methods include contacting a sample comprising a target nucleic acid molecule of interest with at least one single stranded nucleic acid targeting probe that specifically hybridizes to the nucleic acid molecule of interest under highly stringent conditions. The targeting probes generally include a nucleic acid sequence that is from at least about 30 bases in length to about the length of the target nucleic acid molecule of interest. To enable isolation of the targeting probe and hence any nucleic acid specifically bound to the targeting probe, the targeting probe includes one or more capture moieties covalently linked to the targeting probe. The targeting probe(s) are captured via the one or more capture moieties, thereby isolating the target nucleic acid molecule of interest specifically bound to the targeting probe. The targeting probe is designed to specifically bind to the target nucleic acid molecule of interest under denaturing conditions. The length of these probes is selected to maintain specificity of hybridization, and allow the use of extremely stringent hybridization conditions, enabling high specificity of capture while limiting non-specific interactions. In some embodiments, the sample is lysed. In some embodiments, the DNA present in the lysate in sheared. In some embodiments, DNA is sheared while maintaining RNA integrity by a combination of physical shearing, including sonication, and DNA-specific enzymatic digestion, including digestion with DNase I, in buffer conditions conducive to enzymatic digestion.

In some embodiments, the sample is contacted under conditions that prevent non-specific nucleic acid hybridization. These conditions can be established by those of ordinary skill in the art given the disclosure presented herein, and include a combination of low salt for example between about 0M and 1M and high temperature, such as between about 20° C. and 85° C., as well as the presence of various concentrations of chaotropic agents, such as but not limited to, guanidinium thiocyanate, guanidinium hydrochloride, urea, and formamide, or any combination thereof. The relationship between these parameters and their effect on the hybridization stability of nucleic acids of different lengths is described in volumes such as RNA: A Laboratory Manual (Rio, Ares, Hannon, Nielsen 2010) and RNA Methodologies (Farrell 2009), both of which are incorporated herein by reference in their entirety. One of ordinary skill in the art can determine the optimum parameters for such highly stringent hybridization conditions using nothing more than routine experimentation given the guidance presented in the specification.

In some embodiments, the sample is contacted under highly stringent hybridization conditions that prevent non-specific interactions, such as high concentrations of guanidinium thiocyanate, guanidinium hydrochloride, urea, formamide or any combinations thereof.

In some embodiments, the highly stringent hybridization conditions include between about 1M and about 6M urea, such as about 1M, about 1.1M, about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, about 3M, about 3.1M, about 3.2M, about 3.3M, about 3.4M, about 3.5M, about 3.6M, about 3.7M, about 3.8M, about 3.9M, or about 4.0M, about 4.1M, about 4.2M, about 4.3M, about 4.4M, about 4.5M, about 4.6M, about 4.7M, about 4.8M, about 4.9M, about 5.0M, about 5.1M, about 5.2M, about 5.3M, about 5.4M, about 5.5M, about 5.6M, about 5.7M, about 5.8M, about 5.9M, or about 6.0M urea, for example between about 1M and about 2M, about 1.5M and about 3M, about 2M and about 5M, about 2.5M and about 4.5M, about 4.5M and about 6 M urea, and the like. In some embodiments, the highly stringent hybridization conditions include between about 1M and about 5M guanidinium thiocyanate and/or guanidinium hydrochloride, such as about 1M, about 1.1M about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, about 3M, about 3.1M, about 3.2M, about 3.3M, about 3.4M, about 3.5M, about 3.6M, about 3.7M, about 3.8M, about 3.9M, about 4.0M, about 4.1M about, 4.2M, about 4.3M, about 4.4M, about 4.5M, about 4.6M, about 4.7M, about 4.8M, about 4.9M, or about 5.0M guanidinium thiocyanate and/or guanidinium hydrochloride, for example between about 1.5 and about 3.5M, about 1M and about 2M, about 1.5M and about 3M, about 2M and about 5M, about 2.5M and about 4.5M, about 4.5M and about 5M guanidinium thiocyanate and/or guanidinium hydrochloride and the like. In some embodiments, the highly stringent hybridization conditions include between about 10% and about 70% formamide, such as about 10%, about 15%, about 20%, about 25%, about 30%, about 35%, about 40%, about 45%, about 50%, about 55%, about 60%, or about 70% formamide, for example between about 20% and 50% formamide. In some embodiments the highly stringent hybridization conditions have between about 0M and 1M salt, such as about 0.1M, about 0.2M about 0.3M, about 0.4M, about 0.5M, about 0.6M, about 0.7M, about 0.8M, about 0.9M, or about 1.0M salt, such as about 0M and about 1M, about 0.1M, to about 0.2M, about 0.3M to about 0.4M, about 0.5M to about 0.6M, about 0.7M to about 0.8M, or about 0.9M, to about 1.0M salt. In some embodiments, strongly denaturing conditions are carried temperature of between about 20° C. and about 85° C., such as about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., or about 85° C. for example between about 40° C. and about 50° C., 20° C. and about 30° C., 40° C. and about 70° C., 35° C. and about 55° C., 55° C. and about 75° C., 45° C. and about 80° C., 40° C. and about 65° C., or about 40° C. and about 80° C. The stringency of the denaturation can vary according to the application. Typically, the highly stringent hybridization conditions are present for a time period sufficient to limit or otherwise reduce non-specific interaction, and/or reduce secondary structure elements in nucleic acids and/or proteins, while at the same time ensuring high yields of the target. In some embodiments, the sample is contacted with the probe under highly stringent hybridization conditions for about 1 hour to about 24 hours, such as about 1, about 2 about 3, about 4, about 5, about 6, about 7, about 8, about 9, about 10, about 11, about 12 about 13, about 14, about 15, about 16, about 17, about 18, about 19, about 20, about 21, about 22, about 23 or about 24 hours, such as between about 1 and about 10, about 1 and about 15, or about 1 and about 3 hours.

In some embodiments, a capture moiety is included into the targeting probe to enable capture or detection. Thus, in some embodiments the targeting probes that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting nucleic acid probes include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments the specific binding agent is has been immobilized for example on a solid support, thereby isolating the target nucleic molecule of interest. By “solid support or carrier” is intended any support capable of binding a targeting nucleic acid. Well-known supports or carriers include glass, polystyrene, polypropylene, polyethylene, dextran, nylon, amylases, natural and modified celluloses, polyacrylamides, agarose, gabbros and magnetite. The nature of the carrier can be either soluble to some extent or insoluble for the purposes of the present disclosure. The support material may have virtually any possible structural configuration so long as the coupled molecule is capable of binding to targeting probe. Thus, the support configuration may be spherical, as in a bead, or cylindrical, as in the inside surface of a test tube, or the external surface of a rod. Alternatively, the surface may be flat such as a sheet or test strip.

In some embodiments, the disclosed methods are used to deplete a sample of a nucleic acid of interest, for example to deplete the sample of RNA, such as ribosomal RNA which may interfere with particular assays. Thus, in some embodiments the isolated target nucleic acid molecule of interest specifically bound to the targeting probe is removed from the sample, thereby depleting the sample of the target nucleic acid molecule of interest.

In some embodiments, the target nucleic acid is a DNA. In some embodiments, the target nucleic acid is a RNA, such as mRNA, tRNA or lincRNA. In a specific embodiment, the target nucleic acid is a lincRNA. In a specific embodiment, the target nucleic acid is a tRNA. In a specific embodiment, the target nucleic acid is an mRNA.

Targeting probes are disclosed for use in the disclosed methods. In some embodiments, the targeting probes are RNA probes, DNA probes, or hybrid RNA/DNA probes that include about 30 nucleotides in length up to the length of the target nucleic acid that are substantially complementary to the target nucleic acid molecule, and may include probes that represent this entire range of sizes or a subset thereof In some embodiments, the targeting probes are designed to tile the entire sequence of the target nucleic acid, with or without filtering for probes that might cross-hybridize with off-target nucleic acids, with or without overlaps between adjacent probes of any length. In some embodiments, the targeting probes are designed to include complementary sequences to a specific subregion(s) of the target nucleic acid. In some embodiments, a targeting probe does not include a modified nucleotide analogue. In some embodiments, the oligonucleotide probe does not include a locked nucleic acid (LNA) nucleotide.

In embodiments, the total length of the probe, including end linked PCR or other tags, is between about 30 nucleotide and the length of the target nucleic acid. In some embodiments the total length of the probe, including end linked PCR or other tags, is at least about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120.

In some embodiments the total length of the probe, including end linked PCR or other tags, is less then about 2000 nucleotides in length, such as less than about 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 500, 750, 1000, 1250, 1500, 1750, 2000 nucleotides in length or even greater. In some embodiments the total length of the probe, including end linked PCR or other tags, is between about 30 nucleotides and about 250 nucleotides, for example about 90 to about 180, about 120 to about 200, about 150 to about 220 or about 120 to about 180 nucleotides in length.

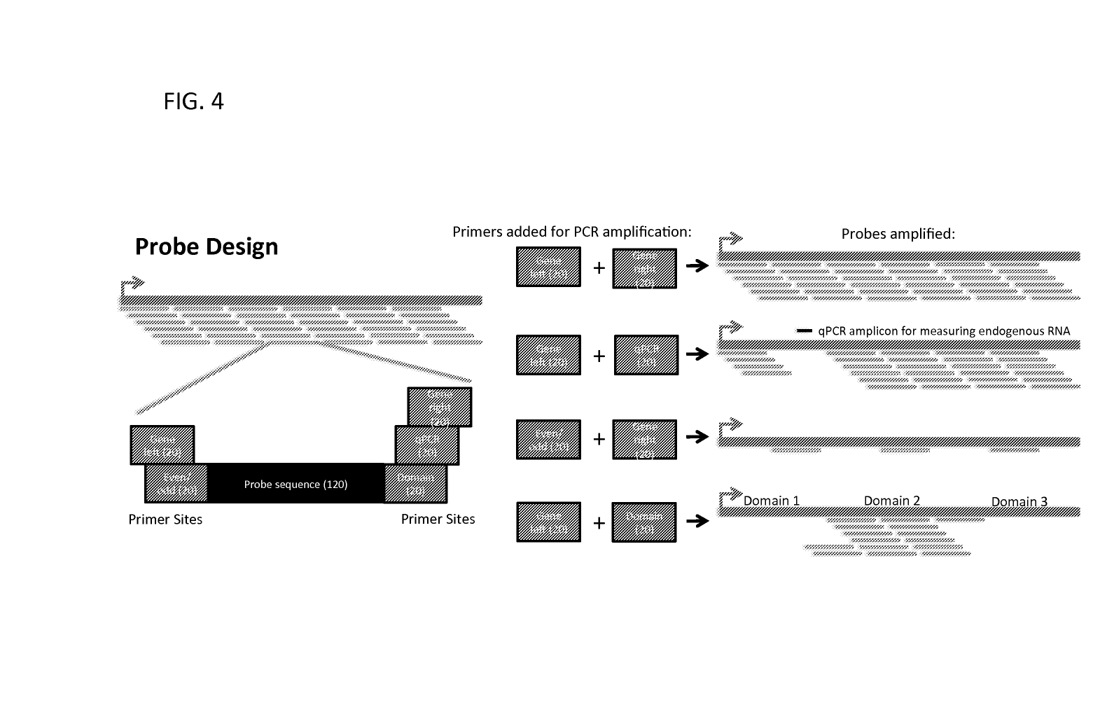

In some embodiments, many possible probesets are synthesized in a pool. Thus, disclosed herein are probeset pools. In some embodiments, these synthesized sequences, or probeset pools contain a hierarchical PCR tag structure that allows for specifically selecting subsets of probes, for example from a pool of synthesized probes. For example with respect to FIG. 4, a targeting probe may include one or more nucleic acid sequence tags, such as PCR tags, that uniquely mark the class, gene, tiling path, and/or domain of the probe, to selectively amplify subsets of these oligos to isolate all probes of a certain class, such as all probes that interact with a specific LincRNA or even related RNAs such as lincRNAs or microRNA that are active in a particular biological pathway. Because the probes in the pool have differing PCR tags, that uniquely mark the class, gene, tiling path, and/or domain etc. of the probe, the present technology makes it possible to select (via amplification) at set of probes having a desired characteristic. In the example where the probes are selected for a specific gene, the probes in the pool can be selected from the pool by PCR amplification using primers to the tag nucleic acid sequences that have been selected to correspond to the particular gene. A similar selection can be made with respect to a particular domain of a gene, for example a particular sequence motif. In such a selection, particular PCR primers are used to amplify the probes that are tagged with a nucleic acid sequence tag used to specify the particular domain. With further reference to FIG. 4, in the example of a gap in the probe set for qPCR measurement, a PCR primer is used that is specific for the “qPCR” tag to selectively amplify a pool of probes that tiles across a target nucleic acid of interest, but leaves a gap in the tiled coverage such that this gap can be used for a qPCR primer/probes or even other probes or primers to bind, for example to determine the levels of the endogenous target RNA in the sample using qPCR. By varying the composition of the forward and reverse PCR primers used, different sets and subsets of probes can be generated from the pool of synthesized probes. Thus, in some embodiments, the targeting probes, comprise specific nucleic acid sequence tags, such as PCR tags, which allows the targeting probes to be selected from a pool of probes specific for different target nucleic acids, and each specific nucleic acid sequence tag, such as a PCR tag, is specific for a specific target nucleic acid. In some embodiments, the targeting probes comprise several different nucleic acid sequence tags, such as PCR tags, for example so that different criteria can be used to select a subset or set of probes from a pool of probes. In some examples, probes targeting a single gene are tagged with a specific nucleic acid sequence tag, such as a PCR tag, for example the probes that are constructed to tile across a gene can be tagged with a specific PCR tag with a unique sequence such that all of the probes targeting the gene can be isolated and/or purified using a nucleic acid complementary to the PCR tag. In addition, relevant subsets of the probes within the gene are tagged, including non-overlapping “even/odd” tiling paths (which enables the use of parallel probesets targeting the same RNA to control for the possibility of nonspecific interactions with the probes themselves), probes targeting one particular domain of the RNA (in order to identify molecules that interact with a that particular domain or subset of the RNA), and probes that leave gaps for qPCR measurement (to allow for measurement of the target RNA without interfering signal from the probes themselves). In some embodiments, the relevant subset of probes targets a set containing multiple (perhaps related) RNA species in order to identify molecules that interact with this entire set. By way of example a specific tag can be used for different probe subsets that target multiple different RNAs. In some embodiments, the targeting probes comprise a series of hierarchical PCR tags such that the same individual probe can be amplified as parts of multiple sets. In some embodiments the targeting probes include one or more specific nucleic acid sequence tags, such as PCR tags. In some examples, the targeting probes include specific nucleic acid sequence tags, such as PCR tags, for distinguishing between different target nucleic acids. In some examples, the targeting probes include specific nucleic acid sequence tags, such as PCR tags, for distinguishing between subregions of the target nucleic acid. In some embodiments, probes are generated by PCR amplification or in vitro transcription of a nucleic acid sequence complementary to the target RNA of interest, followed by shearing of the nucleic acid by some means, including enzymatic, mechanic, or chemical, to target probes of a desired size range, such as between about 30-100 nucleotides, 50-200 nucleotides, 100-1000 nucleotides, or any subset of lengths in which some proportion of the fragments land between 30 and the length of the target RNA.

In some embodiments, a set of probes is used to target a specific target nucleic acid. In some examples, the set of probes is a plurality of single stranded nucleic acid targeting probes, wherein the different single stranded nucleic acid targeting probes in the plurality are complementary to different nucleic acid sequences within the target nucleic molecule of interest. In some examples, the plurality of specific probes of different lengths selected to coordinate with the target nucleic acid molecule of interest. In some examples, the plurality of single stranded nucleic acid targeting probes have a length selected in a distribution about an optimal range.

In some embodiments, the sample is contacted with a plurality of single stranded nucleic acid targeting probes, wherein the different single stranded nucleic acid targeting probes in the plurality are complementary to different nucleic acid sequences within the target nucleic molecule of interest. Because any single region of a target RNA might be unavailable due to the presence of a protein-binding site or strong secondary structure it is advantageous to use multiple probes that target different nucleic acid sequences present in the target nucleic acid sequence. In some embodiments, this is accomplished by tiling the probes across the entire target nucleic acid sequence. In specific embodiments, the targeting probes are spaced with their 5′ and/or 3′ ends spaced between 1 and 50 nucleotides apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, nucleotides apart, for example between 1 and 50, 5 and 45, 10 and 35, 15 and 30, 12 and 50 and the like nucleotides apart. In some examples the probes in the tiled set overlap. In specific embodiments 120-nucleotide probes are tiled across the entire sequence, spaced 15-nt apart, to maximize capture. In some embodiments the targeting probes in the plurality of probes are selected to tile across a target nucleic molecule of interest, for example either with or without overlapping ends.

In some embodiments, the targeting probes are RNA probes, such as RNA probes generated from a DNA oligo pool by incorporating a RNA promotor (such as a T7 promoter) through multiple rounds of PCR. After PCR amplification, in vitro transcription (IVT) with RNA polymerase produces a RNA targeting probe. In some examples, 16-biotin, such as about 25% 16-biotin-uridine is incorporated into the IVT reaction so that the probe is randomly biotinylated throughout its entire length, allowing for rapid probe capture with streptavidin.

The methods disclosed herein are particularly suited to analyzing the molecules, such as nucleic acids and proteins that interact with a target nucleic acid in vivo. In this aspect of the disclosed methods, the sample to be analyzed is contacted with a protein-nucleic acid crosslinking agent, a nucleic acid-nucleic acid crosslinking agent, a protein-protein crosslinking agent or any combination thereof before contacting the sample with a targeting probe. By this method, proteins and/or nucleic acids that interact with a target nucleic acid become crosslinked to the target nucleic acid, such that when the target nucleic acid is isolated, the crosslinked proteins and/or nucleic acids are also isolated as a complex with the target nucleic acid. By altering the types of crosslinkers, individual interaction can be discerned. By this method, primary, secondary and tertiary interactions can be discerned. In some examples, a crosslinker is a reversible crosslinker, such that the crosslinked molecules can be easily separated. In some examples, a crosslinker is a non-reversible crosslinker, such that the crosslinked molecules cannot be easily separated. In some examples, a crosslinker is light, such as UV light. In some examples, a cross linker is light activated. These crosslinkers include formaldehyde, disuccinimidyl glutarate, UV-254, psoralens and their derivatives such as aminomethyltrioxsalen, glutaraldehyde, ethylene glycol bis[succinimidylsuccinate], and other compounds known to those skilled in the art, including those described in the Thermo Scientific Pierce Crosslinking Technical Handbook, Thermo Scientific (2009) as available on the world wide web at piercenet.com/files/1601673_Crosslink_HB_Intl.pdf.

In some embodiments, the captured probes are washed at a temperature above room temperature, for example in a wash buffer, to remove any molecules non-specifically bound to the target nucleic acid, the targeting probe proteins and/or nucleic acids that are crosslinked to the target nucleic molecule of interest, or any combination thereof In some embodiments the captured probes are washed at a selected combination of temperature, denaturing condition, salt concentration, and/or other condition sufficient to substantially remove molecules that non-specifically bind to the targeting probe.

In some embodiments, the captured probes are washed at a temperature of less than about 90° C., such as about 35° C. and about 50° C., such as about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 60° C., about 65° C., about 70° C., about 75° C., about 80° C., about 85° C., or about 90° C., for example between about 35° C. and 55° C. or about 40° C. and about 80° C.

The methods and compositions disclosed herein enable the specific elution of target RNA mediated interactions by elution with specific nucleases, chemicals, or other agents that specifically target dsRNA regions. Thus the methods disclosed herein offer a previously unknown method of determining, at the nucleotide level, the nucleic acid interactions in a system, such as a biological system. In some embodiments, the captured RNAs and/or RNA complexes are eluted from the solid surface, for example off of beads to which streptavidin is attached. In some embodiments, elution includes contacting the captured RNA and/or RNA complex with an agent that targets double stranded nucleic acid regions, such as a double stranded RNA region. In certain embodiments, elution includes contacting the captured probes with specific nucleases, chemicals, or other agents that specifically target double stranded nucleic acid regions. Specific nucleases, chemicals, or other agents that specifically target double stranded nucleic acid regions are known in the art. In some embodiments, elution is done by boiling in a detergent solution, for example in N-laroylsarcosine or sodium deoxycholate solution, such as 1% to about 3% detergent (for example, an about 2% detergent solution) solution, which maintains RNA integrity. In some embodiments, elution is RNase elution with a dsRNA-specific RNase, which increases the specificity of the elution by selecting for complexes where the target RNA is bound to the RNA probe. This procedure elutes proteins by digestion of double stranded RNA, which only releases proteins bound to an RNA that is captured by a biotinylated RNA probe. In some embodiments, eluting includes reverse crosslinking and contacting the probes with proteinase K. In some embodiments, elution includes degrading RNA in high alkaline conditions. In some embodiments, elution comprises specific disruption of the interaction between the probe and the target RNA, for example, heating to break the hybrid between the probe and the target RNA. Other examples include, dsRNA-specific nuclease to digest RNA probes capturing RNA, such as RNase H, digest DNA probes to capture RNA. In some embodiments elution comprises specific disruption of the interaction between the interaction between the probe and the solid support, such as breaking streptavidin-biotin interaction, when using a probe containing biotin, or incorporation into the probe covalently attached to solid support a photo-cleavable linker that is broken through exposure to UV light.

In some embodiments, the methods further include determining the identity of protein and/or nucleic acid crosslinked to the target nucleic acid, for example after elution. Methods of determining the identity of a protein or nucleic acid are known in the art and exemplary methods are given below. In some embodiments, determining the identity of the protein includes the use of an antibody. In some embodiments, determining the identity of the protein includes mass spectrometry. In some embodiments, determining the identity of a nucleic acid comprises sequencing, nucleic acid hybridization and/or PCR.

In some embodiments of the disclosed methods, the proteins that are crosslinked to the target nucleic acid are detected through novel epitopes recognized by polyclonal and/or monoclonal antibodies such as used in ELISA assays, immunoblot assays, flow cytometric assays, immunohistochemical assays, radioimmuno assays, Western blot assays, immunofluorescent assays, chemiluminescent assays and other polypeptide detection strategies (Wong et al., Cancer Res., 46: 6029-6033, 1986; Luwor et al., Cancer Res., 61: 5355-5361, 2001; Mishima et al., Cancer Res., 61: 5349-5354, 2001; Ijaz et al., J. Med. Virol., 63: 210-216, 2001). Generally these methods utilize antibodies, such as monoclonal or polyclonal antibodies.

Generally, protein immunoassays include incubating a sample in the presence of antibody, and detecting the bound antibody by any of a number of techniques well known in the art. The sample can be brought in contact with and immobilized onto a solid phase support or carrier such as nitrocellulose, or other solid support which is capable of immobilizing cells, cell particles or soluble proteins. The support may then be washed with suitable buffers followed by treatment with the antibody. The solid phase support can then be washed with the buffer a second time to remove unbound antibody. If the antibody is directly labeled, the amount of bound label on solid support can then be detected by conventional means. If the antibody is unlabeled, a labeled second antibody, which detects that antibody that specifically binds protein can be used.

In one embodiment, an enzyme linked immunosorbent assay (ELISA) is utilized to detect the protein (Voller, “The Enzyme Linked Immunosorbent Assay (ELISA),” Diagnostic Horizons 2:1-7, 1978, Microbiological Associates Quarterly Publication, Walkersville, Md.; Voller et al., J. Clin. Pathol. 31:507-520, 1978; Butler, Meth. Enzymol. 73:482-523, 1981; Maggio, (ed.) Enzyme Immunoassay, CRC Press, Boca Raton, Fla., 1980; Ishikawa, et al., (eds.) Enzyme Immunoassay, Kgaku Shoin, Tokyo, 1981).

In some examples, the method can include contacting the sample with a second antibody that specifically binds to the first antibody that specifically binds to the peptide or protein. In some examples, the second antibody is detectably labeled, for example with a fluorophore (such as FTIC, PE, a fluorescent protein, and the like), an enzyme (such as HRP), a radiolabel, or a nanoparticle (such as a gold particle or a semiconductor nanocrystal, such as a quantum dot (QDOT®)). In this method, an enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to: malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme.

Detection can also be accomplished by visual comparison of the extent of enzymatic reaction of a substrate in comparison with similarly prepared standards. It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody of the present invention.

Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection can also be accomplished using any of a variety of other immunoassays. For example, by radioactively labeling the antibodies or antibody fragments, it is possible to detect fingerprint gene wild-type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). In another example, a sensitive and specific tandem immunoradiometric assay may be used (see Shen and Tai, J. Biol. Chem., 261:25, 11585-11591, 1986). The radioactive isotope can be detected by such means as the use of a gamma counter or a scintillation counter or by autoradiography.

Proteins also can be detected by mass spectrometry assays coupled to immunoaffinity assays, the use of matrix-assisted laser desorption/ionization time-of-flight (MALDI-TOF) mass mapping and liquid chromatography/quadrupole time-of-flight electrospray ionization tandem mass spectrometry (LC/Q-TOF-ESI-MS/MS) sequence tag of proteins separated by two-dimensional polyacrylamide gel electrophoresis (2D-PAGE) (Kiernan et al., Anal. Biochem., 301: 49-56, 2002; Poutanen et al., Mass Spectrom., 15: 1685-1692, 2001), electrospray ionization (ESI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample can be subjected to one or more dimensions of chromatographic separation, for example, one or more dimensions of liquid or size exclusion chromatography.

Mass spectroscopic methods, such as SELDI, can be used to analyze and identify proteins in a sample. In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest. Chromatographic surfaces can be composed of hydrophobic, hydrophilic, ion exchange, immobilized metal, or other chemistries.

This disclosure also provides integrated systems for high-throughput testing, or automated testing. The systems typically include a robotic armature that transfers fluid from a source to a destination, a controller that controls the robotic armature, a detector, a data storage unit that records detection, and an assay component such as a microtiter dish comprising a well having a reaction mixture for example media.

In some embodiments of the disclosed methods, determining the identity of a nucleic acid includes detection by nucleic acid hybridization. Nucleic acid hybridization involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992).

Other methods for detecting and/or quantifying RNA are well known in the art. In some examples, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, RNA can be reverse-transcribed using a GeneAmp® RNA PCR kit (Perkin Elmer, Calif.), following the manufacturer's instructions. The derived cDNA can then be used as a template in the subsequent PCR reaction or for DNA sequencing.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendable by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments dissociate in solution, and a signal from the released reporter dye is freed from the quenching effect of the second fluorophore. One molecule of reporter dye is liberated for each new molecule synthesized, and detection of the unquenched reporter dye provides the basis for quantitative interpretation of the data.

A variation of RT-PCR is real time quantitative RT-PCR, which measures PCR product accumulation through a dual-labeled fluorogenic probe (e.g., Taqman® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., Genome Research 6:986-994, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems (Foster City, Calif.).

TaqMan® RT-PCR can be performed using commercially available equipment, such as, for example, ABI PRISM 7700® Sequence Detection System® (Perkin-Elmer-Applied Biosystems, Foster City, Calif.), or Lightcycler® (Roche Molecular Biochemicals, Mannheim, Germany). In one example, the 5′ nuclease procedure is run on a real-time quantitative PCR device such as the ABI PRISM 7700® Sequence Detection System®.

In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

In some examples, nucleic acids are identified or confirmed using the microarray technique.

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids.

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

In some embodiments, the identity of a nucleic acid is determined by DNA or RNA sequencing. Generally, the sequencing can be performed using automated Sanger sequencing (AB13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®).

The disclosed methods are also particularly suited to monitoring disease states, such as disease state in an organism, for example a plant or an animal subject, such as a mammalian subject, for example a human subject. Certain disease states may be caused and/or characterized differential binding or proteins and/or nucleic acids to a target nucleic acid in vivo. For example, certain interactions may occur in a diseased cell but not in a normal cell. In other examples, certain interactions may occur in a normal cell but not in diseased cell. Thus, using the disclosed methods a profile of the interaction between a target nucleic acid in vivo, can be correlated with a disease state.

Accordingly, aspects of the disclosed methods relate to correlating the interactions of a target nucleic acid with proteins and/or nucleic acid with a disease state, for example cancer, or an infection, such as a viral or bacterial infection. It is understood that a correlation to a disease state could be made for any organism, including without limitation plants, and animals, such as humans.

The interaction profile correlated with a disease can be used as a “fingerprint” to identify and/or diagnose a disease in a cell, by virtue of having a similar “fingerprint.” The profile of double-stranded DNA binding proteins can be used to identify binding proteins and/or nucleic acids that are relevant in a disease state such as cancer, for example to identify particular proteins and/or nucleic acids as potential diagnostic and/or therapeutic targets. In addition, the profile can be used to monitor a disease state, for example to monitor the response to a therapy, disease progression and/or make treatment decisions for subjects.

The ability to obtain an interaction profile allows for the diagnosis of a disease state, for example by comparison of the profile present in a sample with the correlated with a specific disease state, wherein a similarity in profile indicates a particular disease state.

Accordingly, aspects of the disclosed methods relate to diagnosing a disease state based on interaction profile correlated with a disease state, for example cancer, an inherited or an infection, such as a viral or bacterial infection. It is understood that a diagnosis of a disease state could be made for any organism, including without limitation plants, and animals, such as humans.

Aspects of the present disclosure relate to the correlation of an environmental stress or state with an interaction profile for a target nucleic acid of interest, for example a whole organism, or a sample, such as a sample of cells, for example a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical libraries for agents that modulate interaction profiles, for example that alter the interaction profile from an abnormal one, for example correlated to a disease state to one indicative of a disease free state. By exposing cells, or fractions thereof (such as nuclear extract), tissues, or even whole animals, to different members of the chemical libraries, and performing the methods described herein, different members of a chemical library can be screened for their effect on interaction profiles simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Preparation and screening of combinatorial libraries is well known to those of skill in the art. Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al, Nature, 354:84-88, 1991; PCT Publication No. WO 91/19735), (see, e.g., Lam et al., Nature, 354:82-84, 1991; Houghten et al., Nature, 354:84-86, 1991), and combinatorial chemistry-derived molecular library made of D-and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids, encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl Acad. Sa. USA, 90:6909-6913, 1993), vinylogous polypeptides (Hagihara et al., J. Am. Chem. Soc, 114:6568, 1992), nonpeptidal peptidomimetics with glucose scaffolding (Hirschmann et al., J. Am. Chem. Soc, 114:9217-9218, 1992), analogous organic syntheses of small compound libraries (Chen et al., J. Am. Chem. Soc, 116:2661, 1994), oligo carbamates (Cho et al., Science, 261 :1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, NY., 1989; Ausubel et al., Current Protocols m Molecular Biology, Green Publishing Associates and Wiley Interscience, N. Y., 1989), peptide nucleic acid libraries (see, e.g., U.S. Pat. No. 5,539,083), antibody libraries (see, e.g., Vaughn et al., Nat. Biotechnol, 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, Jan 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produced in a variety of manners including, but not limited to, spatially arrayed multipin peptide synthesis (Geysen, et al., Proc. Natl. Acad. Sa., 81(13):3998-4002, 1984), “tea bag” peptide synthesis (Houghten, Proc. Natl. Acad. Sa., 82(15):5131-5135, 1985), phage display (Scott and Smith, Science, 249:386-390, 1990), spot or disc synthesis (Dittrich et al., Bworg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97 (2):411-448, 1997).

Devices for the preparation of combinatorial libraries are also commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, for example, ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Libraries can include a varying number of compositions (members), such as up to about 100 members, such as up to about 1,000 members, such as up to about 5,000 members, such as up to about 10,000 members, such as up to about 100,000 members, such as up to about 500,000 members, or even more than 500,000 members. In one example, the methods can involve providing a combinatorial chemical or peptide library containing a large number of potential therapeutic compounds. Such combinatorial libraries are then screened by the methods disclosed herein to identify those library members (particularly chemical species or subclasses) that display a desired characteristic activity.

The compounds identified using the methods disclosed herein can serve as conventional “lead compounds” or can themselves be used as potential or actual therapeutics. In some instances, pools of candidate agents can be identified and further screened to determine which individual or subpools of agents in the collective have a desired activity.

Control reactions can be performed in combination with the libraries. Such optional control reactions are appropriate and can increase the reliability of the screening. Accordingly, disclosed methods can include such a control reaction. The control reaction may be a negative control reaction that measures the transcription factor activity independent of a transcription modulator. The control reaction may also be a positive control reaction that measures transcription factor activity in view of a known transcription modulator.

Compounds identified by the disclosed methods can be used as therapeutics or lead compounds for drug development for a variety of conditions. Because gene expression is fundamental in all biological processes, including cell division, growth, replication, differentiation, repair, infection of cells, etc., the ability to monitor transcription factor activity and identify compounds which modulator their activity can be used to identify drug leads for a variety of conditions, including neoplasia, inflammation, allergic hypersensitivity, metabolic disease, genetic disease, viral infection, bacterial infection, fungal infection, or the like. In addition, compounds identified that specifically target transcription factors in undesired organisms, such as viruses, fungi, agricultural pests, or the like, can serve as fungicides, bactericides, herbicides, insecticides, and the like. Thus, the range of conditions that are related to transcription factor activity includes conditions in humans and other animals, and in plants, such as agricultural applications.

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will appreciated that any method of obtaining tissue from a subject can be utilized, and that the selection of the method used will depend upon various factors such as the type of tissue, age of the subject, or procedures available to the practitioner. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

The following examples are provided to illustrate certain particular features and/or embodiments. These examples should not be construed to limit the invention to the particular features or embodiments described.

### EXAMPLES

### Example 1

### Investigating RNA Function

Combined with appropriate crosslinking, RAP provides a powerful tool to investigate RNA function by allowing the enrichment and identification of molecular components—including proteins, DNA loci, and other RNAs—that interact with a target RNA in vivo.

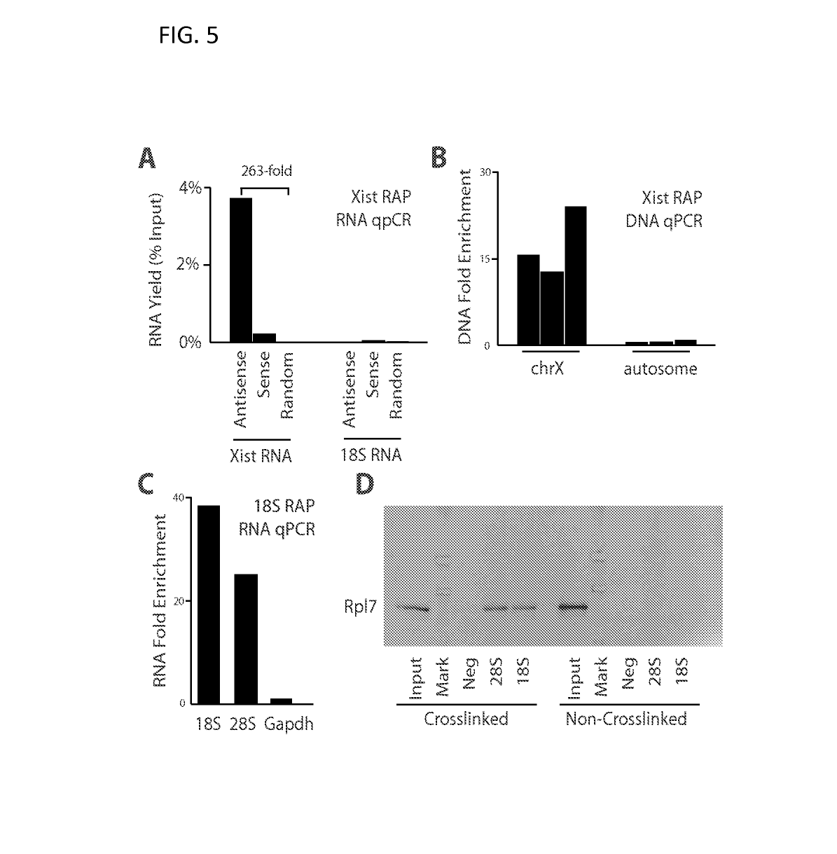

Mouse cells were treated with nucleic acid-protein and protein-protein crosslinkers (formaldehyde+DSG). Xist, a 16 kb RNA that plays a critical role in X-chromosome inactivation in females, was pulled down using targeting probes that specifically bound Xist. A 263-fold enrichment was achieved for Xist RNA compared to sense-probe and random-probe controls, while capturing ˜4% of the input RNA. This result demonstrated that RAP sensitively and specifically selects target RNAs from crosslinked samples (FIG. 5A). The tradeoff between enrichment and yield can be fine-tuned by adjusting the hybridization conditions. Next, enriched DNA was examined by qPCR and it was found that Xist RAP resulted in 15- to 20-fold enrichment of X chromosome loci, known localization sites of Xist (FIG. 5B). Thus RAP can be used for mapping the genomic localization of RNAs on a genome-wide scale.

To examine RNA-RNA and RNA-protein interactions, probes were designed to target mouse 18S ribosomal RNA. 18S RAP was performed in UV-crosslinked cells, where only direct nucleic acid to protein interactions are fixed. Targeting 18S RNA resulted in 39-fold enrichment (FIG. 5C) and 47% yield. A 25-fold enrichment for 28S RNA was found, another component of the ribosome, indicating that RAP can identify other RNAs associated in complexes with the target. Finally, 28S ribosomal RNA was pulled down and a Western blot was performed for Rp17, a ribosomal protein that interacts with 28S. It was found that Rp17 was present specifically in the 28S pulldown but not in a negative control pulldown, showing that RAP enriches for RNA-bound proteins as well (FIG. 5D). In summary, RAP enables an entire suite of assays that can be used to uncover the molecular mechanisms of an RNA

### Example 2

### RNA Selection

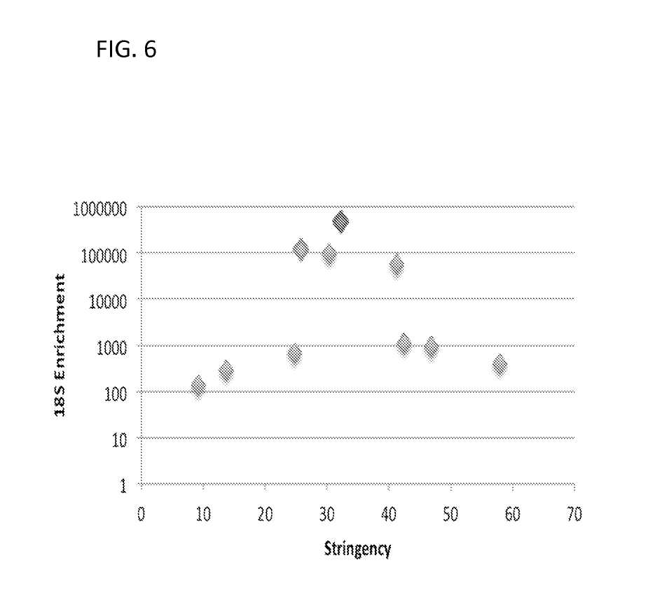

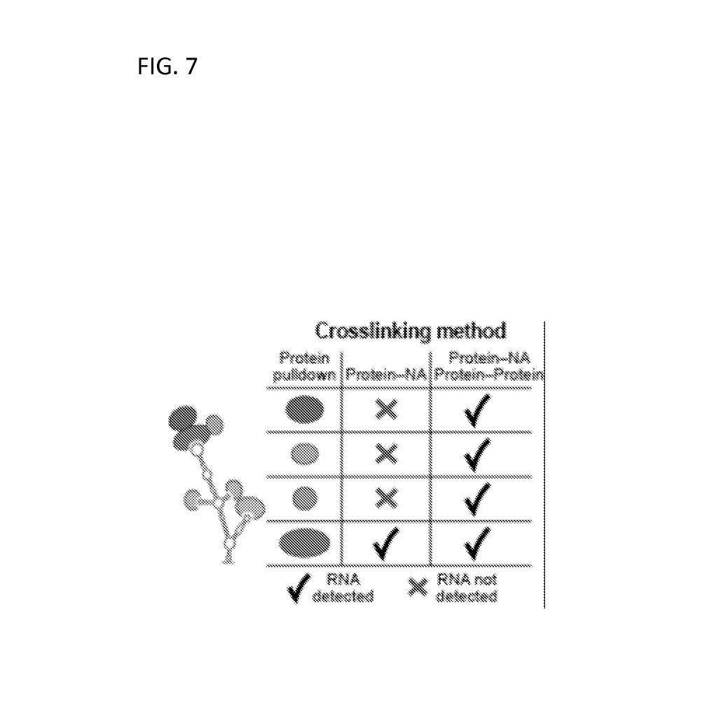

The long RNA probes used in RAP allow for extremely high selection stringency compared to other methods. Indeed, when selecting a target RNA from a purified RNA sample we can increase enrichments to nearly 1,000,000-fold by adjusting hybridization conditions (FIG. 6). This ability is used for capturing RNAs from purified RNA samples for purposes including RNA sequencing to investigate somatic mutations or RNA editing.

### Example 3

### RNA Depletion

In this application, tiled probes are used to capture unwanted RNAs and remove them from a sample. This is particularly useful in RNA-Sequencing applications, in which the sequencing libraries are often overwhelmed by the most abundant RNAs (rRNAs, snRNAs, etc.). Probes are generated against all of the most abundant RNAs in the cell and incubated with purified RNA. This removes any RNA that hybridizes to the probes, decreasing the complexity of the sample and increasing the representation of less abundant RNAs. This method yields >99% depletion of targeted RNAs with no depletion of non-target RNAs. This provides an improvement over poly-A selection, a method typically used for this purpose, because it works in degraded or fragmented RNA samples such as those obtained from clinical derived archived samples (such as Formalin Fixed Paraffin Embedded samples).

### Example 4

### Analysis of Large Intergenic Noncoding RNAs (lincRNAs) Using RAP

The mammalian genome encodes many thousands of large non-coding transcripts including a class of ˜3500 large intergenic ncRNAs (lincRNAs) identified using a chromatin signature of actively transcribed genes. LincRNAs are globally functional in the cell and play roles in diverse biological processes including in the regulation of the pluripotent cell state. While it is now clear that lincRNAs are functionally important, the mechanism by which they carry out their regulatory role is currently unknown. As many of the lincRNAs interact simultaneously with multiple different protein complexes, one hypothesis is that lincRNAs act as ‘flexible modular scaffolds’ to bring together protein complexes into larger functional units. In this model, RNA contains discrete domains that interact with specific protein complexes. These RNAs, through a combination of domains, bring individual regulatory components into proximity resulting in the formation of a unique regulatory RNA. Here we will decipher the mechanism of lincRNA mediated regulation by understanding how lincRNA-Protein complexes form, localize to regulatory targets, and give rise to phenotypic states. Using RAP, protein-complexes with which lincRNAs interact, are identified and the protein interaction sites on RNA map and assembled. These results will provide an understanding of how lincRNAs can utilize discrete domains to target and regulate specific sets of genes and may allow the creation of synthetically engineered RNAs that can carry out engineered regulatory roles.

Understanding the function of each lincRNA requires knowledge of the protein complexes that interact with each lincRNA. The proteins associated with embryonic stem cell (ESC) lincRNAs are determined in vivo RNA affinity purification (RAP) followed by Mass-Spectrometry analysis to selectively enrich specific lincRNAs and identify associated protein complexes that are crosslinked to it.

First, lincRNAs and their crosslinked protein complexes are purified using RAP (FIG. 1). Using long biotinylated antisense RNA probes tiled across the lincRNA sequence allows for the specific and efficient capture a lincRNA. Due to the use of long antisense probes, RAP utilizes denaturing conditions ensuring that proteins that associate with lincRNAs are covalently crosslinked and not forming in solution, a well-known problem for RNA-Protein complexes. Proteins are specifically eluted by digestion of double stranded RNA, which only releases proteins bound to an RNA that is captured by a biotinylated RNA probe. It has been shown that RAP provides enrichments generally exceeding 1000-fold for target RNAs while simultaneously capturing >40% of the endogenous RNA. Moreover, RAP in the presence of in vivo crosslinking, can specifically recover the known proteins associate with an RNA (FIG. 5).

Second, the protein complexes crosslinked to lincRNAs are identified using Mass-Spectrometry analysis. ESCs are crosslinked in vivo using UV and UV plus disuccinimidyl glutarate (UV-DSG), which can distinguish proteins that directly interact with lincRNAs from proteins that interact with lincRNAs through protein intermediates. For each lincRNA, RAP is performed in extracts from UV and UV-DSG crosslinked cells and label purified proteins using isobaric tags (iTRAQ) or Stable Isotope Labeling by Amino acids in Cell culture (SILAC). As a control, RAP is performed with sense probes, which should not pulldown the lincRNA, and label them with compatible iTRAQ tags or SILAC tagged. These four samples will be pooled and quantified using Mass-Spectrometry allowing the identification of specific proteins.

Using RAP, high purity of crosslinked proteins is expected because high levels of enrichment are achieved for target RNAs. In this case, the purified proteins can be characterized with no upstream fractionation steps significantly reducing the cost of Mass-Spectrometry analysis enabling cost-effective characterization of each ESC lincRNA. As a negative control, RAP will be performed in non-crosslinked cells, where no proteins are expected to interact under denaturing conditions allowing us to determine the complexity of each sample and the feasibility of this approach.

Finally, this approach will be used to identify the proteins associated with each ESC lincRNA. RAP is performed on each individual lincRNA and characterize the proteins associated with it. In this case, the RAP method is readily scalable to allow characterization of all ˜250 ESC lincRNAs as (1) the long probes are synthesized on microarrays and selectively labeled allowing synthesis of probes targeting all ESC lincRNAs, control mRNAs, and negative controls on a single array, (2) RAP testing makes use of magnetic separations which can be automated in 96-well plates, (3) the RAP approach provides high yields of target RNAs reducing the number of cells required, and (4) RAP is performed in ESCs where obtaining large numbers of cells is practical.

If the complexity of each sample is high, a pooling approach is used where n ˜20 lincRNA pools are designed, each of which will contain overlapping lincRNAs. If the proteins that interact with each lincRNA are relatively unique, assignment of proteins to each lincRNA is done using computational methods. If the proteins that interact with each lincRNA are relatively similar, then these pools are used to determine the relatively small set of proteins that associate with lincRNAs. ESCs will also be infected with epitope tagged ORFs for each of these proteins and CLIP performed followed by ES lincRNA profiling.

### Example 5

### Determination of LincRNA Localization to Regulatory Targets Using RAP

While it is now clear that lincRNAs have major functional effects in the cell, their direct regulatory targets remain unknown. As many lincRNAs physically interact with diverse regulatory proteins including many chromatin regulators, an attractive hypothesis is that many lincRNAs may work by localizing regulatory proteins to specific genomic DNA regions as has been demonstrated for the XIST ncRNA. Yet, beyond a few examples, these interactions remain largely unexplored. Determining how lincRNAs provide regulatory specificity requires defining the direct targets and the mechanism of lincRNA targeting.

RAP coupled to pooled sequencing of associated DNA regions for all ˜250 ESC expressed lincRNAs is used to evaluate the lincRNAs. Crosslinking methods are used to specifically crosslink only nucleic acids or nucleic acids to protein complexes which will allow us to determine precisely how lincRNAs physically interact with target sites, either through direct nucleic acid hybridization or through protein intermediates. This will allow for the computational definition of common rules governing localization and the combining of this information with maps of lincRNA-Protein complexes to identify common proteins that interact with specific targets. The direct targets of lincRNAs are determined by mapping lincRNA localization sites on genomic DNA using RAP followed by pooled DNA sequencing (RAP-Seq) (FIG. 1). Cells are crosslinked with formaldehyde to capture complex interactions and fix ncRNAs in proximity to genomic DNA. To enable high resolution mapping, DNA is digested into ˜300 bp fragments and lincRNA is pulled down using tiled oligonucleotide pools. To ensure specificity, discrete probe pools are used for each lincRNA. As a control, sense probes are used, which should not pulldown any RNA, but will control for background DNA hybridization. In preliminary results, it is shown that the RAP method enables efficient capture of endogenous ncRNAs in the presence of in vivo crosslinking and that by pulling down the XIST ncRNA, we can achieve specific recovery of distinct genomic regions on the X chromosome but not autosomes (FIG. 4).

Each is of the ˜250 ES lincRNAs is mapped. The RAP-Seq method is readily scalable as (1) it can be performed in 96-well plates using automation from probe generation to sequencing library preparation and (2) many samples (>24) can be multiplexed in a sequencing lane at current yields.

Understanding the role of lincRNAs in localizing complexes to regulatory targets requires knowledge of how lincRNAs achieve specificity. If lincRNAs localize to genomic DNA or RNA targets, there are two potential models, (1) lincRNAs can directly base pair with nucleic-acid target sites or (2) lincRNAs can interact with protein complexes to localize to target sites. Using differential crosslinking methods and performing RAP-Seq, distinguishes between localization models at each genomic site.

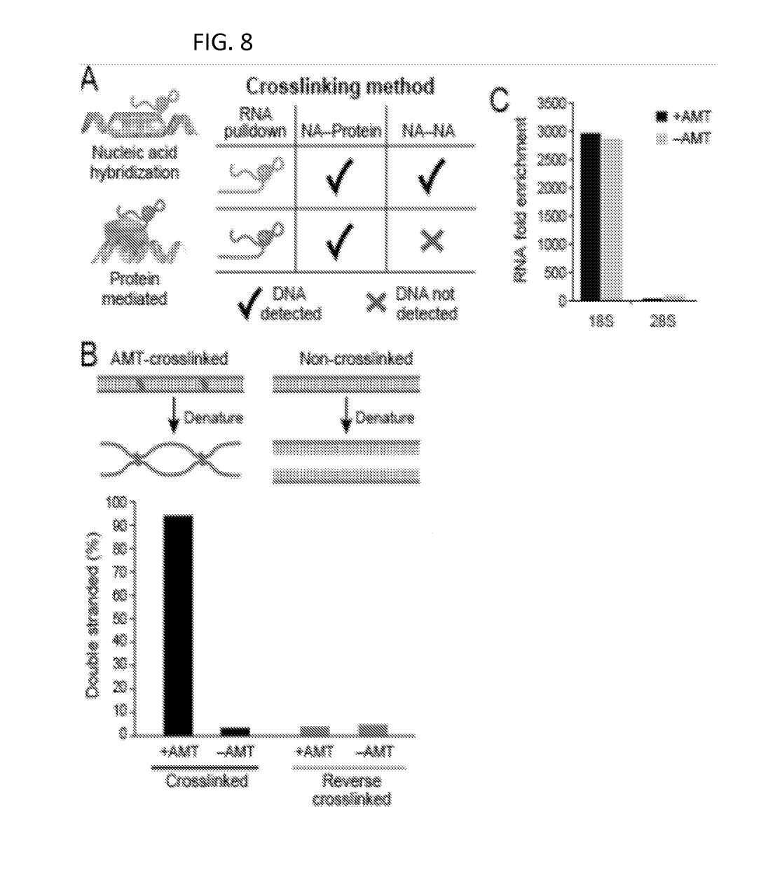

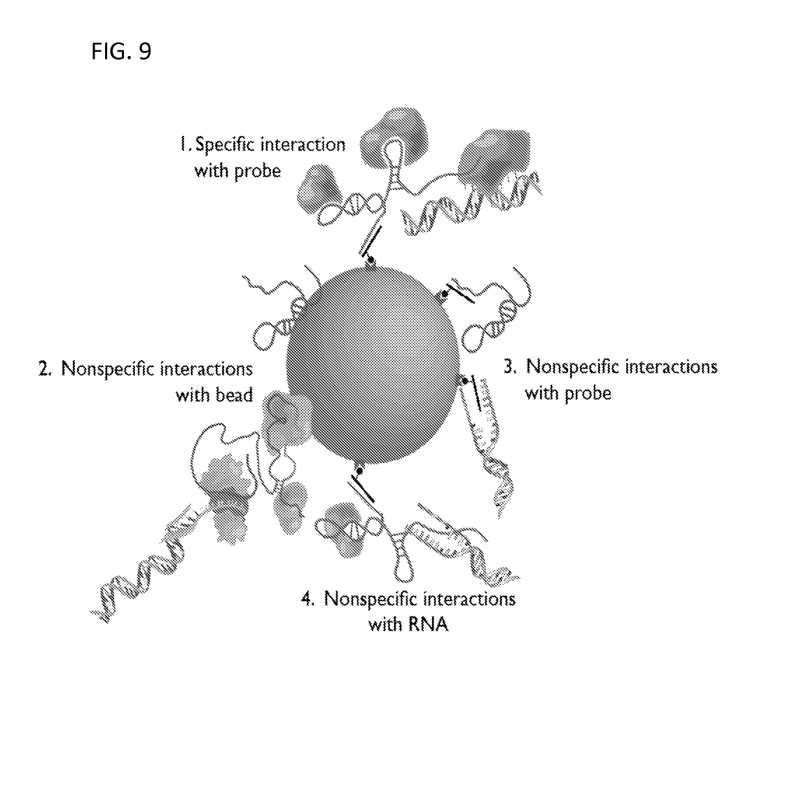

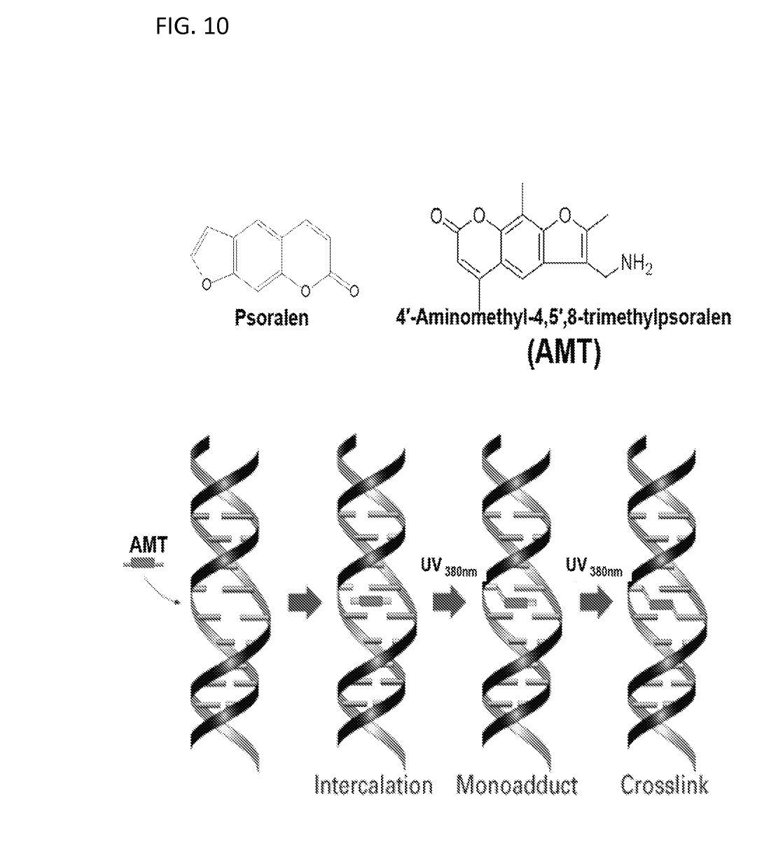

Interacting nucleic acids are crosslinked using psoralen derivatives, which have been shown to reversibly crosslink interacting nucleic acid molecules in vivo. As nucleic acids are crosslinked directly, all trials can be performed after digesting proteins to ensure the specificity of interacting nucleic acids enabling identification of genomic DNA regions that directly base pair with a lincRNA (FIG. 8). In preliminary results, it was found that aminomethyltrioxalen (AMT), a psoralen derivative, can be added directly to ESCs and provides efficient in vivo crosslinking of nucleic acids where >90% of interacting nucleic acids are crosslinked every 50 nucleotides. Moreover, endogenous RNA can be captured and the crosslinks reversed to sequence the associated DNA (FIG. 8).

RAP is performed in AMT crosslinked cells for the ˜250 ES lincRNAs and sequence the associated DNA regions. This AMT data will augment the data generated in formaldehyde crosslinked cells which fixes protein complexes to nucleic acids. As a positive control, RAP is performed in AMT and formaldehyde cells on the telomerase ncRNA (Terc) which directly base pairs with genomic DNA. This is compared to RAP on the XIST ncRNA which interacts with genomic DNA through protein intermediates.

To identify direct nucleic acid interactions, the AMT and formaldehyde datasets are compared. This will provide a map of lincRNA interactions that occur through nucleic acid or through protein mediated interactions.

### Example 6

### Protocol for lincRNA Probe Generation

The following protocol was used for the generation of lincRNA probes and can be adapted for the generation of any targeting probes as disclosed herein. In addition, while specific times and reagents are specified, it is contemplated that different albeit similar reagents and times and temperatures can be employed by those of ordinary skill in the art with minimal experimentation, given the guidance presented herein.

In vitro Transcription for Approximately 10 μg Yield

A. The IVT reaction was set up as shown in the following table:

| IVT Mix | 1 Reaction |
|---|---|
| DNA template (~250 ng) + H₂O | 11.3 μl |
| 10× T7 Transcription Buffer | 2 μl |
| 20 mM ATP | 1 μl |
| 20 mM CTP | 1 μl |
| 20 mM GTP | 1 μl |
| 20 mM UTP | 0.75 μl |
| 10 mM 16-Biotin UTP | 0.5 μl |
| T7 RNA Polymerase | 2 μl |
| 100 mM DTT | 0.25 μl |
| RNase Inhibitor | 0.2 μl |
| Total | 20 μl |

Mix well by pipetting.
Incubate at 37° C. overnight (4+ hours).

B. DNase Treatment
Denature RNA/DNA hybrids by incubating at 85° C. for 3 minutes.
Place immediately on ice for 1 min.
Add 21 μl H₂O, 5 μl 10× TURBO™ DNase Buffer, and 4 μl TURBO DNase (50 μl total volume). (TURBO™ DNase works better than DNase I at high salt concentrations, although other DNases can be used in the this protocol).
Incubate at 37° C. for 15 minutes.

C. RNeasy® Mini Cleanup
Add 175 μl (3.5×) RLT. Add 1.5× (337.5 ul) 100% ethanol.
Mix, and transfer (562.5 ul) to an RNeasy® Mini spin column.
Spin 15 s at >8,000×g. Discard flow-through.
Add 500 μl Buffer RPE and spin for 15 s at >8,000×g. Discard flow-through.
Add 500 μl Buffer RPE and spin for 2 min at >8,000×g.
Transfer to new collection tube and spin once more to remove carryover Buffer RPE.
Transfer column to 1.5 mL tube. Add 50 μl H₂O and spin lmin at >8,000×g to elute.

D. Quality Control
NanoDrop® to determine yield.
Run products on gel (e.g. Lonza RNA Flash Gel) to visualize sizes (should be a single band at 171 nucleotides; usually there is some small smear below this size representing partial length product).
   lincRNA Probe Generation (˜100 ug Yield)

A. The IVT reaction was set up as shown in the following table:

| IVT Mix | 1 Reaction |
|---|---|
| DNA template (~500 ng) + H₂O | 19.7 μl |
| 10× T7 Transcription Buffer | 4 μl |
| 100 mM ATP | 2 μl |
| 100 mM CTP | 2 μl |
| 100 mM GTP | 2 μl |
| 100 mM UTP | 1.5 μl |
| 10 mM Labeled UTP | 5 μl |
| T7 RNA Polymerase | 3 μl |
| 100 mM DTT | 0.4 μl |
| RNase Inhibitor | 0.4 μl |
| Total | 40 μl |

B. DNase Treatment
Denature RNA/DNA hybrids by incubating at 85° C. for 3 minutes.
Place immediately on ice for 1 min.
Add 21 μl H₂O, 5 μl 10× TURBO™ DNase Buffer, and 4 μl TURBO DNase (50 μl total volume). (TURBO™ DNase works better than DNase I at high salt concentrations, although other DNases can be used in the this protocol)
Incubate at 37° C. for 15 minutes.

C. RNeasy® Mini Cleanup
Add 350 μl (3.5×) RLT. Add 1.5× (675 μl) 100% ethanol.
Mix, and transfer (700 μl at a time) to two RNeasy® Mini spin columns.
Spin 15 s at >8,000×g. Discard flow-through.
Add 500 μl Buffer RPE and spin for 15 s at >8,000×g. Discard flow-through.
Add 500 μl Buffer RPE and spin for 2 min at >8,000×g.
Transfer to new collection tube and spin once more to remove carryover Buffer RPE.
Transfer column to 1.5 mL tube. Add 50 μl H₂O and spin 1 min at >8,000×g to elute.

D. Quality Control
NanoDrop® to determine yield. (When using all unlabeled nucleotides, this protocol yields 75 ug of RNA, or 1.36 nmol; expect less when incorporating labeled nucleotides).
Run products on gel (e.g. Lonza RNA Flash Gel) to visualize sizes (should be a single band at 171 nucleotides; usually there is some small smear below this size representing partial length product).

### Example 6

### RNA Pulldown from Nuclear Lysates

The following protocol was used to pull down RNA from a nuclear lysate and can be adapted for the generation of any targeting probes as disclosed herein. In addition, while specific times and reagents are specified, it is contemplated that different albeit similar reagents and times and temperatures can be employed by those of ordinary skill in the art with minimal experimentation, given the guidance presented herein.

A. Starting material
For each RNA pulldown perform a sense and antisense control.
Start with nuclear lysates from ˜500K cells per pulldown.

B. Preclear lysate
Preclear pooled lysate.
Take 3× volume Streptavidin C1 beads per sample (7.5 μL).
Wash 2× in Hybridization buffer.
Resuspend in 7.5 μl of hybridization buffer.
Add 7.5 μl of washed beads to ˜100 uL of lysate.
Incubate at 45° C. for 30 minutes.
Magnetically separate and transfer to clean tube (2×).
Pool lysates.

C. Hybridization
Save 2× 5% (5 μL) of precleared lysate as input (5% for DNA and 5% for RNA), Freeze at −80° C.
Dilute 10 ng each biotin-coupled RNA oligo into 2 μL water in strip tubes.
Denature probes at 85° C. for 3 minutes then place immediately on ice.
Combine lysate and probe, and immediately transfer to a 45° C.
Thermocycler.
Incubate for 2 hours.

D. Prepare Streptavidin beads
Mix beads well (vortex).
Take 2.5 μl of Invitrogen C1 Streptavidin beads per reaction.
Place beads on magnet for 1-2 min and remove supernatant.
Wash beads 2× in GuSCN Hyb Buffer.
Resuspend beads in 2.5 μl of 3M GuSCN Hyb buffer.
Add to hybridization mix.

E. Binding hybrid to beads
Transfer washed Streptavidin beads to hybrids (2.5 μl per reaction).
Incubate at 45° C. for 15 min on Thermal Mixer at 1400 rpm.

F. Nucleic acid washes
Preheat Wash Buffers at 55° C.
Magnetically separate and remove supernatant.
(If using large volume: resuspend in 100 μl of 3M GuSCN wash buffer and transfer to strip tube).
Wash 2× 45° C. with 100 μL 3M GuSCN wash buffer for 5 minutes.
Wash 2× 50° C. with 100 μL 3M GuSCN wash buffer for 5 minutes.
Wash 2× 55° C. with 100 μl 3M GuSCN wash buffer for 5 minutes.
After last wash, take 10 μL of beads for RNA analysis.
Transfer remaining 90 μL to new strip tube.

G. RNA Analysis: Elution
Retrieve Input samples from freezer. Add 30 μL NLS Elution Buffer, 2.25 μL 5M NaCl, 3.75 μL ProK, 14 μL H₂O.
Take 10 μL of washed beads and elute 2× in 15 μL NLS Elution Buffer at 94° C. for 5 min.
Combine eluates and place immediately on ice.
Add 2.25 μL 5M NaCl, 3.75 μL ProK, 24 μL H2O.
Incubate at 65° C. for 1-2 hours.

H. DNA Analysis: Elution and Reverse Crosslinking
Retrieve Input samples from freezer. Add 66 μL of NLS Elution Buffer, 4 μL 5M NaCl, 4 μL ProK).
Remove and discard final wash.
Add 76 μL NLS Elution Buffer, 4 μL 5M NaCl, 4 μL ProK to each sample. Incubate overnight at 65° C. in the thermal cycler.

I. RNA Analysis: SPRI Clean-up
Add 2.2× volume of SPRI Beads (132 μl).
Mix well by pipetting and incubate at RT for 2 minutes.
Place on magnet for 4 minutes.
Remove supernatant.
Add 100 μl of 70% EtOH while on magnet.
Incubate for 30 seconds and remove EtOH.
Remove plate from magnet and allow to dry at RT for 5 minutes.
Elute in 17 μL of water, do not remove from beads.

J. QC: RNA analysis
To each RNA sample, add 1 μl TURBO® DNase, 20 μl 10× TURBO® DNase buffer.
Incubate 37° C. for 15 minutes.
Clean up using 2.2× SPRI beads (44 μL), elute in 22 μl H₂O.
Freeze at −80° C.

| cDNA Mix | 1X |
|---|---|
| 10X Random primers | 2 ul |
| 10X AffinityScript Buffer | 2 ul |
| 100 mM DTT | 2 ul |
| 100 mM dNTP | 0.8 ul |
| AffinityScript RT Enzyme | 1 ul |
| Total | 7.8 ul |

### Reverse Transcription

Add 7.8 μl of cDNA mix to 12.2 μl of RNA, mix well.

Incubate at 25 C for 10 minutes, 55° C. for 1 hour, 70° C. for 15 minutes.

qPCR

Dilute cDNA 1:10 (add 180 ul H₂O)

| qPCR Mix | 1X |
|---|---|
| Primer mix (25 uM) | 1.5 ul |
| Roche Sybr mix | 28.5 ul |
| Diluted cDNA | 30 ul |

qPCR Mix 1×

### DNA Library Prep

K. Aliquot Samples
Transfer samples off beads.
Take 40 μl to cleanup.
Freeze ˜44 μl in −80° C. as backup.

L. SPRI Cleanup
Add 2.2× volume of SPRI Beads (88 μl).
Mix well by pipetting and incubate at RT for 2 minutes.
Place on magnet for 4 minutes.
Remove supernatant.
Add 100 μl of 70% EtOH while on magnet.
Incubate for 30 seconds and remove EtOH.
Remove plate from magnet and allow to dry at RT for 5 minutes.
Elute in 16.6 μl of water, leave on bead.

M. End Repair

Make a mix consisting of:

End Repair Mix 1 Reaction 6.6 Reactions

| End Repair Mix | 1 Reaction | 6.6 Reactions |
|---|---|---|
| dsDNA | 16.6 ul | |
| NEBNext End Repair Buffer | 2 ul | 13.2 ul |
| NEBNext End Repair Enzymes | 1.5 ul | 6.6 ul |
| Total | 20 ul | |

Incubate at 20° C. for 30 minutes.

N. End Repair SPRI Cleanup
Add 2.2× volume of SPRI Beads (44 μl).
Mix well by pipetting and incubate at RT for 2 minutes.
Place on magnet for 4 minutes.
Remove supernatant.
Add 100 μl of 70% EtOH while on magnet (2×).
Incubate for 30 seconds and remove EtOH.
Leave plate on magnet and allow to dry at RT for 4-5 minutes (or until dry).
Add 16.5 μl of H₂O.

O. dA Tailing

| dA-Tailing Mix | 1 Reaction | 6.6 Reactions |
|---|---|---|
| dsDNA | 16.5 ul | |
| 10X dA-Tailing Buffer | 2 ul | (add individually) |
| Klenow Fragment | 1.5 ul | (add individually) |
| Total | 20 ul | |

Incubate at 37° C. for 50 minutes

P. dA-Tailing SPRI Cleanup
Add 2.2× volume of SPRI beads (44 μl).
Mix well by pipetting and incubate at RT for 2 minutes.
Place on magnet for 4 minutes.
Remove supernatant.
Add 100 μl of 70% EtOH while on magnet (2×).
Incubate for 30 seconds and remove EtOH.
Leave plate on magnet and allow to dry at RT for 4-5 minutes (or until dry).
Add 20 μl of EB (do not remove from beads).

Q. Adaptor Ligation

Use 1:10 adaptor concentration, where 1:1 is the concentration in a new plate.

| Adaptor Ligation Mix | 1 Reaction | 8.8 Reactions |
|---|---|---|
| dsDNA + H₂O | 20 ul | |
| 5x Quick Ligation Buffer | 6 ul | (add individually) |
| DNA Adaptors 1:10!!! | 0.8 ul | (add individually) |
| Quick T4 DNA Ligase | 3.3 ul | (add individually) |
| Total | 30 ul | (add 32 ul mix) |

AdIncubate at 20 C for 50 minutes

R. Adaptor SPRI Cleanup
Add 18 μl of SPRI to 30 μl of ligation mix.
Mix well by pipetting and incubate at RT for 2 minutes.
Place on magnet for 4 minutes, Remove supernatant.
Add 100 μl of 70% EtOH while on magnet (2×).
Incubate for 30 seconds and remove EtOH (2×).
Leave plate on magnet and allow to dry at RT for 4-5 minutes (or until dry)
Elute in 50 μl EB, mix well, let stay 2-5 min.

S. REPEAT Adaptor SPRI Cleanup
Add 45 μl of SPRI to 50 μl of eluted DNA.
Mix well by pipetting and incubate at RT for 2 minutes.
Place on magnet for 4 minutes, Remove supernatant.
Add 100 μl of 70% EtOH while on magnet (2×).
Incubate for 30 seconds and remove EtOH (2×).
Leave plate on magnet and allow to dry at RT for 4-5 minutes (or until dry).
Elute in 50 μl H₂O. Save 25 μL (half) in −80° C. as Pre-PCR Backup.

T. PCR Enrichment

Make a mix consisting of:

| PCR Mix | 1 Reaction |
|---|---|
| dsDNA | 24 ul |
| Primer Mix (25 uM?) | 1 ul |
| 2× Phusion MasterMix | 25 ul |
| Total | 50 ul |

Run using PCR program ChIP-NEB:

| | | | | |
|---|---|---|---|---|
| Initial Denaturation | 98 C. | 30 | Seconds | 1 cycle |
| Denaturation | 98 C. | 15 | seconds | |
| Annealing | 68 C. | 30 | seconds | 1 cycle |
| Extension | 72 C. | 30 | seconds | |
| Denaturation | 98 C. | 12 | seconds | |
| Annealing | 59 C. | 20 | seconds | 1 cycle |
| Extension | 72 C. | 30 | seconds | |
| Denaturation | 98 C. | 12 | seconds | |
| Annealing | 68 C. | 20 | seconds | 12 cycle |
| Extension | 72 C. | 20 | seconds | |
| Final Extension | 72 C. | 1 | minute | 1 cycle |

U. Enrichment SPRI Cleanup
Add 0.9× SPRI Beads (45 μl).
Mix well by pipetting and incubate at RT for 2 minutes.
Place on magnet for 4 minutes.
Remove supernatant.
Add 100 μl of 70% EtOH while on magnet (2×) Incubate for 30 seconds and remove EtOH (2×)
Leave plate on magnet and allow to dry at RT for 4-5 minutes (until dry).
Add 50 μl of H2O.

V. Enrichment SPRI Cleanup
Add 0.9× SPRI Beads (45 μl).
Mix well by pipetting and incubate at RT for 2 minutes.
Place on magnet for 4 minutes.
Remove supernatant.
Add 100 μl of 70% EtOH while on magnet (2×) Incubate for 30 seconds and remove EtOH (2×).
Leave plate on magnet and allow to dry at RT for 4-5 minutes (until dry)
Add 21 μl of H₂O.
Place on Magnet, use 1 μl for DNA High Sensitivity bioanalyzer chip

W. QC Libraries
Quantify dsDNA concentrations using Qubit®

### Example 7

### ssDNA Probe Generation, Reverse Transcription Method

Yield is ˜3-6 ug ssDNA probe at the end of the two pages, depending on the target.

Yield for IVT reaction (first step) should be ˜50 ug (1 RNeasy column).

A. In vitro Transcription

Set up IVT reaction:

| IVT Mix | 1 Reaction |
|---|---|
| T7 DNA template (~125 ng) + H₂O | 12.1 μl |
| 10× T7 Transcription Buffer | 2 μl |
| 100 mM ATP | 1 μl |
| 100 mM CTP | 1 μl |
| 100 mM GTP | 1 μl |
| 100 mM UTP | 1 μl |
| T7 RNA Polymerase | 1.5 μl |
| 100 mM DTT | 0.2 μl |
| RNase Inhibitor | 0.2 μl |
| Total | 20 μl |

Mix well by pipetting.
Incubate at 37° C.>4 hours or overnight.

B. DNase Treatment
Denature RNA/DNA hybrids by incubating at 85° C. for 3 minutes.
Place immediately on ice for 1 min.
Add 21 μl H₂O, 5 μl 10× TURBO DNase Buffer, and 4 μl TURBO DNase (50 μl total volume). (TURBO DNase works better than DNase I at high salt concentrations)
Incubate at 37° C. for 15 minutes

B. RNeasy Mini Cleanup
Add 175 μl (3.5×) RLT. Add 1.5× (337.5 μl) 100% ethanol.
Mix, and transfer (562.5 μl) to an RNeasy Mini spin column.
Spin 15 s at >8,000×g. Discard flow-through.
Add 500 ul Buffer RPE and spin for 15 s at >8,000×g. Discard flow-through.
Add 500 ul Buffer RPE and spin for 2 min at >8,000×g.
Transfer to new collection tube and spin once more to remove carryover Buffer RPE.
Transfer column to 1.5 mL tube. Add 30 ul H₂O and spin 1 min at >8,000×g to elute.

C. Quality Control
NanoDrop to determine yield and dilute to a convenient concentration (e.g., 1 μg/μL)
Optional: Run products on gel (e.g. Lonza RNA Flash Gel) to visualize sizes (should be a thick band around 175 nucleotides; there is a +/−5 nt smear because the T7 polymerase does not start precisely at the same nucleotide every time; usually there is also some faint smear below this size representing partial length product).

D. Reverse Transcription
CAREFUL: Combine the correct primer with the correct RNA. Using our current nomenclature (where the “18S AS” probe targets the 18S sense strand and thus will not capture the RNA), combine “18S” RNA template with the biotinylated 18S-R-enrich primer, and combine “18S-AS” RNA template with the biotinylated 185-L-enrich primer.
Mix RNA and biotinylated primer together. Denature at 75° C. for 5 minutes, then switch to 55° C. in incubator. Remove reaction and immediately add RT mix.

| RT Reaction | 1 Reaction |
|---|---|
| RNA template (10 μg) + H₂O | 120 μl |
| Biotinylated primer (100 μM) | 20 μl |
| 10X AffinityScript Buffer | 20 μl |
| 100 mM DTT | 20 μl |
| 100 mM dNTP | 8 μl |
| AffinityScript RT Enzyme | 10 μl |
| RNase Inhibitor | 2.5 μl |
| Total | 200 μl |

Incubate at 55° C. for 50 minutes, then 75° C. for 5 minutes.
Add 0.1× (20 μl) 1M NaOH and 0.02× (4 μl) 0.5M EDTA.
Incubate at 75° C. for another 10 minutes.
Add 0.1× (20 μl) 1M acetic acid to neutralize.
Optional: Run 1 μl of reaction on RNA FlashGel. Should see two bands: 1 for primer and 1 for product.

E. Clean-up
Clean with Zymo RNA Concentrator-5 columns using the following protocol:
Add 2× volume RNA Binding Buffer (˜480 μl) and mix well.
Add 1.9× original volume 100% ethanol (456 μl) and mix well.
Transfer half volume to each of two Zymo columns.
Spin at >12,000×g for 1 minute. Discard flowthrough.
Add 400 μl RNA Prep Buffer. Spin and discard flowthrough.
Add 700 μl RNA Wash Buffer. Spin and discord flowthrough.
Repeat the wash step with 400 μl RNA Wash Buffer.
Remove flowthrough and spin once more for 2 minutes.
Transfer column to a 1.5 mL tube. Elute in 30 μl of water by spinning at 10,000×g for 1 minute.
Measure concentration with NanoDrop.

Switch sense/antisense sample labels. (e.g., “18S” RNA template generates “18S-AS” ssDNA probe).

### Example 8

### RAP with DNA Probes

Goal: Generate production quality data for RAP-RNA methods paper

Input: V6.5 s+DMSO
V6.5 s+Spliceostatin
V6.5 s+DMSO, crosslinked for 10 minutes with 3% FA-DSG together
V6.5 s no crosslinking

Probes: Malat1 (Jan. 28, 2014 JE), U1 (pool of three from IDT), Fine Even/Odd ˜Jan. 15, 2014 JE

### RAP-DNA

| | | | |
|---|---|---|---|
| 1) | Malat1 | DMSO | 5M |
| 2) | U1 | DMSO | 5M |
| 3) | Malat1 | SSA | 5M |
| 4) | U1 | SSA | 5M |
| 5) | Malat1 | NC (5M vol) | 2.5M |
| 6) | U1 | NC (5M vol) | 2.5M |
| 7) | Malat1 | DMSO-Xlink | 5M |
| 8) | Firre-Odd | V6.5 | 5M |
| 9) | Firre-Even | V6.5 | 5M |
| 10) | Input-DMSO | | |
| 11) | Input-SSA | | |
| 12) | Input-NC | | |
| 13) | Input-DMSO-Xlink | | |
| 14) | Input-V6.5 | | |

### RAP-RNA

| | | | |
|---|---|---|---|
| 1) | Malat1 | DMSO | 5M |
| 2) | U1 | DMSO | 5M |
| 3) | Malat1 | SSA | 5M |
| 4) | U1 | SSA | 5M |
| 5) | Malat1 | NC (5M vol) | 2.5M |
| 6) | U1 | NC (5M vol) | 2.5M |
| 7) | Input-DMSO | | |
| 8) | Input-SSA | | |
| 9) | Input-NC | | |

Use Axygen 1.7 mL low-retention tubes

A. Preclear lysate

Preclear pooled lysate

Take 200 uL Streptavidin C1 beads per 5M cells

Wash 2× in the same volume of Hybridization Buffer.

Resuspend in 0.5× volume of Hybridization Buffer

Add washed beads to lysate.

Incubate at 37° C. for 20 minutes

Magnetically separate and transfer to clean tube (2×)

Pool lysates if applicable

B. Hybridization

Save 50K cells precleared lysate as input (10 uL). Save on ice

Pre-heat lysate before adding probes.

Dilute DNA probes into 2 uL water in strip tubes,

Denature probes at 85° C. for 3 minutes then place immediately on ice.

Combine lysate and probe, and immediately transfer to a 37° C. thermomixer.

Incubate for 2.5 hours shaking.

C. Prepare Streptavidin beads

Mix beads well (vortex)

Take 125 nL of Invitrogen C1 Streptavidin beads per 500 ng probe (625 nL for this experiment)

Place beads on magnet for 1-2 min and remove supernatant

Wash beads 2× in Hybridization Buffer

Resuspend beads in 0.25× volume of Hybridization Buffer

D. Binding hybrid to beads

Transfer washed Streptavidin beads to hybridization reaction.

Incubate at 37° C. for 30 min on Thermal Mixer at 1400 rpm

E. Wash beads

Preheat Wash Buffers at 45° C.

Magnetically separate and remove supernatant.

Wash 3× 45° C. with 1× bead volume No Salt Wash Buffer for 6 minutes

Wash 3× 47° C. with 1× bead volume No Salt Wash Buffer for 6 minutes

F. RNase Elution

Wash 1× with 1× bead volume RNase H Elution Buffer (add TCEP fresh).

Wash 1× with 100 μl RNase H Elution Buffer (add TCEP fresh).

Transfer to new tube before removing final wash.

Following volumes are for 625 uL of streptavidin beads:

Add 50 uL RNase Elution Buffer, 7.5 uL RNase H, 5 uL RNase Cocktail (A and T1)

Incubate at 37° C. for 30 minutes on Thermal Mixer at 1200 rpm.

Remove and save the eluate.

Add 62.5 uL Hybridization Buffer and incubate at 37° C. for 5 minutes shaking

Remove eluate and combine with previous eluate.

Magnetically separate the combined eluates once more and transfer to a new tube (remove residual beads and attached DNA probe)

Add 312.5 uL NLS Elution Buffer, 50 uL 5M NaCl, 12.5 uL ProK.

Total volume for 625 uL/2.5 ug samples: 500 uL

Input samples: Add 77.5 uL of NLS Elution Buffer, 10 uL 5M NaCl, 2.5 uL ProK)

Seal 1.7 mL tubes with paraffin.

Incubate overnight at 60° C. in the thermal mixer, shaking

For RAP-RNA samples, incubate at 65° C. for 1.5 hours, then clean and freeze or proceed to RNA library prep.

G. SILANE Cleanup

Cool samples on ice before SILANE clean-up.

Add 50 uL SILANE beads in 50 uL 5M NaCl. Add 1× isopropanol (550 uL).

Wash 2× in 500 uL 70% EtOH

Dry for 10 minutes.

Elute in 25 uL H₂O and remove from beads.

Add 5 uL SILANE beads in 87.5 uL RLT. Add 1× isopropanol (122.5 uL).

Wash 2× in 100 uL 70% EtOH

Dry for 5 minutes.

Elute in 12.5 uL H₂O.

NEBNext Ultra Library Prep (from 10-50 pg of input DNA)

H. End Repair

| End Repair | 1 Reaction |
|---|---|
| dsDNA | 12.5 μl |
| End Repair Buffer 10x | 1.5 μl |
| End Prep Enzyme Mix | 1 μl |
| Total | 15 μl |

Incubate at 20° C. for 30 minutes.

Add 10 mM NaCl(1 μl of 160 mM) to each sample and mix.

Incubate at 55° C. for 30 minutes. Hold at 4° C.

I. Adapter Ligation

| Adapter Ligation | 1 Reaction |
|---|---|
| End Repair Reaction | 15 μl |
| Blunt/TA Ligase Master Mix | 3.75 μl |
| Ligation Enhancer | 0.25 μl |
| 1:10 Dilution of NEB Adapter | 1 μl |
| Total | 20 μl |

Incubate at 20° C. for 45 minutes.

Add 1 μL of USER enzyme. Mix and incubate at 37° C. for 15 minutes.

Add 19 μl H₂O to 40 μl total.

J. Adapter Clean-up

Add 0.7× volume SPRI (Ampure XP) beads.

Mix thoroughly and wait 3 minutes.

Place on magnet. Separate.

Wash 2× with 100 μl of 70% EtOH, moving tube on magnet so beads fly from side to side.

Remove ethanol and wait 8 minutes or until dry.

Add 40 μl H₂O at end, leaving beads in the tube.

Repeat clean-up with 1× volume SPRI beads. Elute in 24 μl H₂O, remove 23 μL from beads.

K. PCR

| PCR Mix | 1 Reaction |
|---|---|
| dsDNA | 23 ul |
| Barcoded Primer Mix (25 μM each) | 2 ul |
| 2× NEBNext High Fidelity MM | 25 ul |
| Total | 50 ul |

| | | | | |
|---|---|---|---|---|
| Initial Denaturation | 98 C. | 30 | Seconds | 1 cycle |
| Denaturation | 98 C. | 15 | seconds | |
| Annealing | 67 C. | 30 | seconds | 4 cycles |
| Extension | 72 C. | 30 | seconds | |
| Denaturation | 98 C. | 15 | seconds | 10 cycles |
| Extension | 72 C. | 30 | seconds | |
| Final Extension | 72 C. | 2 | minutes | 1 cycle |

L. PCR SPRI Cleanup

Clean with 1× volume SPRI beads. Elute in 15 μl H₂O.

Buffers

| | Final (M | Stock (M | 1X | 1.4X |
|---|---|---|---|---|
| Hybridization Buffer | or %) | or %) | (mL) | (mL) |
| 20 mM Tris-HCl (pH 7.5) | 0.02 | 1 | 1 | 1.4 |
| 7 mM EDTA | 0.007 | 0.5 | 0.7 | 0.98 |
| 3 mM EGTA | 0.003 | 0.5 | 0.3 | 0.42 |
| 150 mM LiCl | 0.15 | 8 | 0.9375 | 1.3125 |
| 1% NP-40 | 1 | 10 | 5 | 7 |
| 0.2% N-lauroylsarcosine | 0.2 | 20 | 0.5 | 0.7 |
| 0.125% Na-Deoxycholate | 0.125 | 4 | 1.5625 | 2.1875 |
| 3M Guanidinium | 3 | 6 | 25 | 35 |
| Thiocyanate | | | | |
| 2.5 mM TCEP | 0.0025 | 0.5 | 0.25 | 0.35 |
| H2O | | | 14.75 | 0.65 |
| | | Total | 50 | 50 |
| | | (mL) | | |

| | Final (M | Stock (M | 1X |
|---|---|---|---|
| No Salt Wash Buffer | or %) | or %) | (mL) |
| 20 mM Tris pH 7.5 | 0.02 | 1 | 1 |
| 10 mM EDTA | 0.01 | 0.5 | 1 |
| 0.1% Na-Deoxycholate | 0.1 | 4 | 1.25 |
| 0.2% N-lauroylsarcosine | 0.2 | 20 | 0.5 |
| 1% NP-40 | 1 | 10 | 5 |
| 3M GuSCN | 3 | 6 | 25 |
| 2.5 mM TCEP | 0.0025 | 0.5 | 0.25 |
| H2O | | | 16 |
| | | Total | 50 |
| | | (mL) | |
| | Final (M | Stock (M | 1X |
| High Salt Wash Buffer | or %) | or %) | (mL) |
| 20 mM Tris pH 7.5 | 0.02 | 1 | 1 |
| 10 mM EDTA | 0.01 | 0.5 | 1 |
| 1M LiCl | 1 | 8 | 6.25 |
| 0.1% Na-Deoxycholate | 0.1 | 4 | 1.25 |
| 0.2% N-lauroylsarcosine | 0.2 | 20 | 0.5 |
| 1% NP-40 | 1 | 10 | 5 |
| 3M GuSCN | 3 | 6 | 25 |
| 2.5 mM TCEP | 0.0025 | 0.5 | 0.25 |
| H2O | | | 9.75 |
| | | Total | 50 |
| | | (mL) | |

| | Final (M | Stock (M | 1X |
|---|---|---|---|
| NLS Elution Buffer | or %) | or %) | (mL) |
| 20 mM Tris pH 7.5 | 0.02 | 1 | 1 |
| 10 mM EDTA | 0.01 | 0.5 | 1 |
| 2% N-lauroylsarcosine | 2 | 20 | 5 |
| 2.5 mM TCEP | 0.0025 | 0.5 | 0.25 |
| H2O | | | 42.75 |
| | | Total | 50 |
| | | (mL) | |

| | Final (M | Stock (M | 1X |
|---|---|---|---|
| Rnase H Elution Buffer | or %) | or %) | (mL) |
| 50 mM Tris pH 7.5 | 0.05 | 1 | 2.5 |
| 75 mM NaCl | 0.075 | 5 | 0.75 |
| 3 mM MgCl2 | 0.003 | 1 | 0.15 |
| 0.125% N-lauroylsarcosine | 0.125 | 20 | 0.3125 |
| 0.025% sodium deoxycholate | 0.025 | 4 | 0.3125 |
| 2.5 mM TCEP | 0.0025 | 0.5 | 0.25 |
| H2O | | | 45.725 |
| | | Total | 50 |
| | | (mL) | |

### Example 9

Volumes listed are for 20M cells per RNA target (10M sense, 10M antisense)

### Probe/Bead Ratio

1M cells

0.5 ug probe

100 ul SA beads

### Cell Lysis

Resuspend pellet in 450 uL ice cold 500 mM LiCl lysis buffer per 1×10⁷ cells

*Use 900 uL lysis buffer for each tube of 20M cells

To each 20M cell tube—

Add 1× protease inhibitor cocktail (4.6 ul of 200× PIC), 23 ul murine RNase inhibitor fresh without EDTA.

10 minutes on ice. Pass through needle if lysate is viscous.

Sonicate 2× with Branson sonicator using microtip, 10% for 30 s (0.7 s on, 1.3 s off)

Add 4.7 ul 200× DNase Salt Stock (0.5M MgCl₂+0.1M CaCl₂) and 15 ul Turbo DNase; 15 min at 37° C.

Return sample to ice and immediately quench DNase by adding EDTA to 10 mM (19.6 ul) and EGTA to 5 mM (9.8 ul). Add TCEP to 2.5 mM (4.9 ul)

Add 500 mM LiCl/6 M urea buffer to final conc. of 4 M urea and let stand 10 min (1962.6 ul 6M urea buffer to 981.3 ul lysate)

Spin down lysate at 10 min at max speed (1° C.)

Transfer supernatant to fresh tube.

*Concentration is 6794 cells/uL lysate.

### Preclear Lysate

Take 1× volume Streptavidin C1 beads (4 mL per 20 M cells)

Split into 2×1.5 mL microfuge tubes for 10M each sense/AS

Wash beads 2× in 1 mL of 500 mM LiCl/4 M Urea buffer (Hyb buffer with EDTA)

Remove last wash

Add lysate to beads, incubate at 55° C. for 30 minutes with shaking at 1000 rpm Magnetically separate and transfer supernatants to clean tubes twice

*Pool lysate samples; take and freeze 50,000 cell inputs (7.4 uL) for RNA QC

*Remove 100,000 cell input for Western blot (14.7 uL)

Hybridize Probe and Target

Denature 5 ug of each probe per 10M cells at 85° C. for 3 minutes, then place immediately on ice.

Combine lysate+probe, and immediately transfer to a 55° C. Thermomixer.

Incubate for 2 hours with mixing at 1000 rpm (15 s on, 15 s off)

Prepare Streptavidin Beads

Take 2 mL of Invitrogen C1 Streptavidin beads per 10 M cell sample

Split into 3×1.5 mL microfuge tubes

Place beads on magnet for 1-2 min and remove supernatant

Wash beads 2× in 1 mL of 500 mM LiCl/4 M Urea buffer (Hyb buffer with EDTA)

Remove Last Wash

*Remove input plus probe sample before mixing lysate with beads −50,000 cells (7.4 uL)

Bind Hybrids to Beads

Transfer hybrids (lysate) to washed Streptavidin beads to microfuge tubes

Incubate at 55° C. for 20 min on Thermomixer at 1200 rpm

*Save flowthroughs for RNA analysis

Wash Hybrids on Beads

Wash 3× with 500 uL of 500 mM LiCl+4M urea HYB buffer for 5 min at 55° C.

Before removing last wash, transfer 5% of beads to a fresh tube for RNA analysis (25 ul of 500 ul is 5%)

Elute from Beads

For RNA QC Samples:
Magnetically separate and remove last wash sup from 5% sample of beads
Add 20 ul NLS pH 8.2 elution buffer (with 10 mM TCEP, 10 mM EDTA)
Heat to 95° C. for 2 minutes. Save supernatant.

For Protein Samples—Benzonase Elution:

Remove last wash and replace with 500 uL of Benzonase Elution Buffer

Add 1.1 uL of benzonase (diluted 1:10) to each 10M cell tube

Incubate for 2 h at 37° C.

Remove supernatant and add 80% TCA to final concentration of 10%

Incubate overnight at 4° C. to precipitate proteins

Protein Analysis

Sample Prep for SDS-PAGE

Prepare input and FT samples:

Dilute to 30 ul with 10 mM Tris buffer, pH 7.5 and add 10 ul of 4× loading dye

Incubate samples at 95° C. for 10 min

TCA precipitation of elution samples:

Precipitate proteins by adding TCA to 10% final concentration

Incubate at 4° C. overnight

Pellet protein by centrifugation, 30 min at max speed at 4° C. in microfuge

Discard supernatant and wash pellet with 1 mL cold acetone

Centrifuge 15 min at max speed

Remove acetone and allow pellets to dry approx. 5 min in fume hood

Resuspend each pellet in 20 uL of 10 mM Tris buffer, pH 7.5

*If pellet is yellow, add 1M Tris base to adjust pH

Add 4× LDS or Licor loading dye plus buffer to bring final volume to 40 uL

Incubate samples at 95° C. for 10 min

SDS-PAGE Bolt 4-12% Bis-Tris Gel (1.5 mm×10 well)
1) Prepare vertical gel chamber by filling with 1× MES-SDS running buffer
2) Load 40 ul sample per well
3) Load SeeBlue Plus2 Ladder as needed (8 ul)
4) Run until dye front reaches bottom of the gel

Transfer to Membrane
1) Make transfer buffer with 10% methanol)
2) Soak 2 pieces Whatman paper and 1 piece nitrocellulose membrane in buffer
3) Take apart gel, cut off combs and foot
4) Sandwich in Novex Xcell transfer chamber or iBlot
5) Transfer 1 hr at 30 volts (room temp) or iBlot 7 min

Total Protein Membrane Stain
1) Apply Blot FASTstain according to manufacturer's instructions (GBiosciences)
2) Destain with 3 washes of warm water (˜5 min total)

Blocking
1) Rinse membrane in PBS (handle with tweezers)
2) Block with Licor blocking buffer/PBS (1:1 mix) for 30 min at RT

Primary Antibodies
1) Make dilutions of primary antibodies in 10 ml PBS+0.1% Tween (anti-rpS6 1:1000, anti-actin 1:400)
2) Incubate for 2 h at RT with slow shaking on rocker table, or ON at 4° C.

Washes and Secondary Antibody
1) Wash membrane 4× 5 min in PBS+0.1% Tween
2) Secondary Licor antibodies incubation (45-60 min) in PBS-T—protect from light (anti-mouse IRDye 680 1:15,000, anti-rabbit IRDye 800 1:20,000)
3) Wash membrane 4× 5 min in PBS-T

Development on LiCor
(1) Develop on Licor with 800 nm GREEN and 680 nm RED channels
(2) Visualize bands and quantify if needed

RNA Analysis

Proteinase K Digestion

Dilute all samples to 20 ul final volume with 2% NLS elution buffer

Add 2.5 ul Proteinase K to each 20 ul sample

Incubate at 55° C. for 1 hr

Perform standard Silane cleanups with 20 ul beads/sample, 10 ul elutions

Silane cleanup 1 (volumes for 20 uL sample):
for each sample to be cleaned, remove liquid from 20 uL silane beads and resuspend in 3× sample vol RLT (for 20 ul sample use 60 ul RLT)
transfer 60 ul beads in RLT to each 20 ul sample
add 1.5× new volume of 100% ethanol (120 ul) alternatively, can use 1× vol of EtOH to exclude smaller fragments
wait 5 min for sample to bind to beads
magnetically separate and wash 2× with 70% ethanol
remove supernatant and air dry beads for ˜5 minutes
elute in 26 uL of 1× TurboDNase buffer—let stand 10 minutes for elution
   can also incubate 2-3 min at 50° C. to encourage elution
leave beads in tube and perform DNase digestion in same tube

DNase Digestion:
For each sample:

| | |
|---|---|
| 26 uL | elution |
| 1 uL | RNase inhibitor |
| 3 uL | DNase |
| 30 uL | total |

Incubate 20 minutes at 37° C.

Silane Cleanup 2 (vol for 30 ul):
add 90 uL RLT to sample and mix
add 180 uL 100% ethanol
wash 2× with 70% ethanol
remove supernatant and allow to air dry for about 5 minutes
elute in 10 uL of 10 mM Tris pH 7.5 and let stand ˜10 min
magnetically separate and transfer supernatant to fresh tube
use this material for BioAnalyzer Pico 6000 or RT reaction

*Run Pico chip on 1 ul samples to check RNA integrity

Reverse Transcription Master Mix:

| | |
|---|---|
| cDNA Mix | 1X |
| H2O | 5.2 ul |
| 10X AffinityScript Buffer | 2 ul |
| 100 mM DTT | 2 ul |
| 100 mM dNTP | 0.8 ul |
| AffinityScript RT Enzyme | 1 ul |

Denature RNA at 70 C for 3 min, put on ice

Add 1 ul 10× Random Primers (9 mers) to each 8 ul RNA sample

Incubate for 10 min at room temperature (annealing)

Add 11 ul Master Mix to each sample

Incubate at 55° C. for 1 hour, 70° C. for 15 minutes (Christine's folder “rt” program)

qPCR Analysis

Dilute cDNA as needed (1:6-1:8 is standard)

| | |
|---|---|
| qPCR Mix | 1X |
| Primer mix (25uM) | 1.5 ul |
| Roche Sybr mix | 27 ul |
| Diluted cDNA | 25.5 ul |

Primers:

Use long primers, when available, to reduce background from probe

Oct Four, GAPDH (loading controls)

Check: (1) enrichment (sense over antisense)
(2) yield (sense over input)

Buffer Recipes:

| | 500 mM | | | | 500 mM | |
|---|---|---|---|---|---|---|
| | LiCl | | 500 mM | | LiCl + | |
| | Lysis | | LiCl + | | 4M urea | |
| Stock Solutions | buffer | | 6M urea | | (Hyb) | |
| 1M Tris pH 7.4 | 100 | ul | 200 | ul | 400 | ul |
| 0.5M EDTA | — | | 200 | ul | 400 | ul |
| 8M LiCl | 625 | ul | 1250 | ul | 2500 | ul |
| 10% Triton X- | 500 | ul | 1000 | ul | 2000 | ul |
| 100 | | | | | | |
| 20% SDS | 100 | ul | 200 | ul | 400 | ul |
| 4% Sod Doc | 250 | ul | 500 | ul | 1000 | ul |
| 8M Urea | — | | 15000 | ul | 20000 | ul |
| 0.5M TCEP | — | | 100 | ul | 200 | ul |
| H2O | 8425 | ul | 1550 | ul | 13100 | ul |
| TOTAL | 10 | mL | 20 | mL | 40 | mL |

Lysis Buffer (no urea, no EDTA): 10 mM Tris pH 7.4, 500 mM LiCl, 0.5% Triton X-100, 0.2% SDS, 0.1% sodium deoxycholate

| Benzonase Elution Buffer | 1X (10 mL) |
|---|---|
| 1M Tris pH 8.0 | 200 ul |
| 2 mM MgCl₂ | 20 ul |
| 20% N-laroylsarcosine | 250 ul |
| 0.5M TCEP | 10 ul |
| H2O | 9520 ul |
| Total | 10,000 ul |

RAP benzonase elution buffer:

20 mM Tris pH 8.0, 0.05% NLS, 0.5 mM TCEP, 2 mM MgCl₂

### Example 10

### AMT Crosslinking with Nuclear Fractionation

Generate AMT-crosslinked RNA from nuclear fraction. Use same crosslinking protocol as Patrick used previous for Sep. 7, 2012 samples. Do a +AMT and −AMT control

Use cell fractionation protocol from Amy PJ, optimized for ES cells Aug. 30, 2013 by JE.

A. Crosslink Cells

Starting Material: 1 15-cm² plate of V6.5 cells (˜20M cells)
1. Trypsinize and pellet cells as usual
2. Gently resuspend in 0.5 mL N2B27 10 times with a 1 mL pipet.
3. Dilute cells to max volume for the tube in PBS, then re-centrifuge (330 g)
4. Make a 1 mg/mL solution of AMT in H₂O by resuspending at 37° C., then dilute 1:2 in 2× PBS. Chill on ice. Protect from light.
5. Resuspend cells in 2.5 mL ice-cold AMT solution (or ice-cold PBS alone for −AMT control). Incubate on ice for 15 minutes. Protect from light.
6. Transfer samples to a pre-chilled 10-cm² dish, tipping plate to disperse cells. Place on ice.
7. Place in Stratalinker (long wave UV 350 nm bulbs) directly under the bulbs—cells should be 3-4 cm from light source.
8. Crosslink for 7 minutes. Mix the cells every 2-3 minutes. AMT will yellow with UV exposure, while the −AMT control will not.
9. Transfer irradiated cells to cold eppendorf tubes.
10. Spin at 330 g for 4 minutes to pellet cells.

B. Cell Fractionation
11. From this point onwards, KEEP CELLS ON ICE AND BUFFERS COLD. Do not pipette cells to resuspend. Instead, gently flick the tubes to get the cells/isolated nuclei back into solution.
12. The following protocol is for 5M cells. Increase volumes/split into more tubes if necessary.
13. To lyse the cytoplasmic/plasma membrane, add 200 uL ice-cold NP-40 lysis buffer (10 mM Tris-HCl [pH 7.5], 0.05% NP-40, 150 mM NaCl) to the cell pellet. Flick to resuspend. Incubate on ice for 5 min.
14. Cut the end off a p200 tip, and gently pipette up the cell lysate over layer the cell lysate over 2.5 volumes (in this instance, 500 uL) of chilled sucrose cushion (24% RNAse-free sucrose in lysis buffer) and centrifuge for 10 min, 4° C., 15000×g in an Eppendorf 5415C centrifuge (This is the max speed for this model) [Note: At this step, too the nuclei can lyse if the centrifugal speed is too high. Please take care to calculate your RCF if using a centrifuge with a larger rotor radius].
15. You should see an opaque/white-ish band of cytoplasmic contents at the top of the supernatant. Collect and save 750 μL of the supernatant (cytoplasmic fraction).
16. Gently add ˜200 ul ice-cold 1× PBS/1 mM EDTA to the nuclei pellet, taking care not to dislodge the pellet. [The aim is to rinse the surface of the pellet to remove any leftover cytoplasmic extract]. Aspirate the PBS/EDTA.
17. Resuspend nuclei pellet in 100 μL of pre-chilled 2× Low Salt Nuclei Buffer (40 mM Tris pH 7.5, 100 mM KCl, 3 mM MgCl₂, 4 mM TCEP—add fresh, 5% v/v RNase inhibitor—add fresh) by gently flicking the tube.
18. Add an equal volume (100 μL) of cold 2× Detergent Mix (2% NP-40, 0.8% Sodium deoxycholate, 0.2% N-lauroylsarcosine). Vortex vigorously for 5 seconds.
19. Incubate on ice for 5 minutes.

Extract Crosslinked RNA
20. Add Mg/Ca DNase mix, 50 μL TURBO DNase
21. Incubate at 37° C. for 20 minutes.
22. Add EDTA/EGTA solution.
23. Add Proteinase K and 0.5% SDS to both nuclear and cytoplasmic fractions and incubate at 37° C. for 1 hour.
24. Clean with phenol-chloroform.
25. Add 1× volume acid phenol chloroform (pH 4.5). Vortex, wait 2 minutes.
26. Spin at max speed (16,000×g) for 10 minutes at 4° C.
27. Take aqueous layer—add 1× volume chloroform. Vortex, wait 2 minutes.
28. Spin at max speed for 10 minutes at 4° C.
29. Take aqueous layer. Repeat chloroform extraction if necessary.
30. Clean with Zymo RNA-25 column with 2× volume RNA Binding Buffer. (Should do ethanol precipitation here instead, or RNeasy column due to large amount of RNA).
31. Spec to determine yield. qPCR or Bioanalyzer to quantify the fractionation.

### Example 11

### snRNA RAP-RNA

Pull down U1 and Malat1 in psoralen crosslinked RNA. Use higher amounts of cells, lower fragmentation time to increase yield and fragment size.

Input: AMT-crosslinked titration from JE Nov. 24, 2013. Use 10 ug input RNA of +/−AMT for each sample.

Fragment RNA: Fragment 20 ug RNA in 200 μL reaction-2.25 min at 70° C.

DNase treat: Add 24 μL DNase buffer, 15 μL TURBO DNase, 1 μL 1M DTT (240 μL total) Clean up with 2× Zymo RNA-25, elute in 50 μL H₂O for each column. Measure concentration on NanoDrop. View un-fragmented and fragmented RNA on Bioanalyzer.

Probes: Biotinylated ssDNA oligo ordered from IDT Sep. 9, 2013 (U1). Use 1 μg probe per tube (5.5 μL of 10 μM oligo solution). Malat1: use ssDNA probes, 4 μg.

| | | |
|---|---|---|
| 15) | U1 snRNA | +AMT |
| 16) | Malat1 | +AMT |
| 17) | U1 snRNA | −AMT |
| 18) | Malt1 | −AMT |
| 19) | Input | +AMT |
| 20) | Input | −AMT |

A. Hybridization

Input samples: Save 20 ng of RNA for input

Dilute DNA probes into 2 uL water in strip tubes,

Denature probes at 85° C. for 3 minutes then place immediately on ice.

Denature input RNA at 85° C. for 3 minutes then place immediately on ice.

Dilute input RNA in 1.5 mL Hybridization Buffer. Heat before adding probe.

Add probe and immediately transfer to 52° C. thermomixer.

Incubate for 2 hours.

B. Prepare Streptavidin Beads

Mix beads well (vortex)

Take 20 μL of Invitrogen C1 Streptavidin beads per 20 ng probe (1 mL for this experiment)

Place beads on magnet for 1-2 min and remove supernatant

Wash beads 2× in Hybridization Buffer

Resuspend beads in 0.25× volume of Hybridization Buffer

C. Binding Hybrid to Beads

Transfer washed Streptavidin beads to hybridization reaction.

Incubate at 52° C. for 30 min on Thermal Mixer at 1200 rpm

D. Wash Beads

Preheat Wash Buffers at 60° C.

Magnetically separate and remove supernatant.

Wash 3× 57° C. with 1 mL Low Stringency NA Wash Buffer for 6 minutes

Wash 3× 57° C. with 1 mL High Stringency NA Wash Buffer for 6 minutes

Transfer to new tube before removing final wash.

E. RNase H Elution

Wash 1× with 1× bead volume RNase H Elution Buffer (add TCEP fresh).

Wash 1× with 500 mL RNase H Elution Buffer (add TCEP fresh).

Transfer to new tube before removing final wash.

Following volumes are for 1 mL of streptavidin beads:

Add 80 uL RNase Elution Buffer, 16 uL RNase H

Incubate at 37° C. for 30 minutes on Thermal Mixer at 1200 rpm.

Remove and save the eluate.

Add 100 nuL Hybridization Buffer and incubate at 37° C. for 5 minutes shaking

Remove eluate and combine with previous eluate.

Magnetically separate the combined eluates once more and transfer to a new tube (remove residual beads and attached DNA probe)

F. SILANE Cleanup

Cool samples on ice before SILANE clean-up.

Add 50 uL SILANE beads in 600 μL RLT. Add 1× ethanol (800 μL).

Wash 2× in 1 mL 70% EtOH

Dry for 8 minutes or until dry.

Elute in 75 uL H₂O. Remove from beads.

Proceed to RNA sequencing. (use 3× size FNK buffer reaction)

Buffers

| | Final (M | Stock (M | 1X | 2X (mL) |
|---|---|---|---|---|
| LiCl Hyb/Lysis Buffer | or %) | or %) | (mL) | no urea |
| 10 mM Tris-HCl (pH 7.5) | 0.01 | 1 | 0.5 | 1 |
| 1 mM EDTA | 0.001 | 0.5 | 0.1 | 0.2 |
| 500 mM LiCl | 0.5 | 8 | 3.125 | 6.25 |
| 1% Triton X-100 | 1 | 100 | 0.5 | 1 |
| 0.2% SDS | 0.2 | 20 | 0.5 | 1 |
| 0.1% Na-Deoxycholate | 0.1 | 4 | 1.25 | 2.5 |
| 4M Urea | 4 | 8 | 25 | |
| H2O | | | 19.025 | 38.05 |
| | | Total | 50 | 50 |
| | | (mL) | | |

| NA High Stringency | Final (M | Stock (M | 1X | 2X (mL) |
|---|---|---|---|---|
| Wash Buffer | or %) | or %) | (mL) | no urea |
| 0.1X SSPE | 0.1 | 20 | 0.25 | 0.5 |
| 0.1% SDS | 0.1 | 20 | 0.25 | 0.5 |
| 1% NP-40 | 1 | 10 | 5 | 10 |
| 4M Urea | 4 | 8 | 25 | |
| H2O | | | 19.5 | 39 |
| | | Total | 50 | 50 |
| | | (mL) | | |
| NA Low Stringency | Final (M | Stock (M | 1X | 2X (mL) |
| Wash Buffer | or %) | or %) | (mL) | no urea |
| 1X SSPE | 1 | 20 | 2.5 | 5 |
| 0.1% SDS | 0.1 | 20 | 0.25 | 0.5 |
| 1% NP-40 | 1 | 10 | 5 | 10 |
| 4M Urea | 4 | 8 | 25 | |
| H2O | | | 17.25 | 34.5 |
| | | Total | 50 | 50 |
| | | (mL) | | |

FNK Buffer (50 mL): 5.8 mL H₂O, 2.5 mL 1M Tris pH 7.5, 250 μL 1M MgCl₂, 1.25 mL 2M KCl, 500 μL 1M DTT, 50 μL 10% Triton X-100.

Directly add 25 μl of master mix:
10 ul 5× FNK buffer
1 uL RNase Inhibitor
3 uL FASTAP
3 uL T4 PNK
0.3 uL 100 mM CaCl₂
1 uL TURBO DNase
1 uL Exonuclease I
5.7 uL H₂O

Incubate at 37° C. for 30 minutes.

Clean up RNA with 10 μL SILANE beads: Add 3× RLT, and 1× isopropanol

Magnetically separate; discard sup, then wash twice with 70% ethanol.

Elute in 6 uL

Add 1 μL of 1 pg/μL Human SOX2 lncRNA probe spike

A. FIRST LIGATION (RNA/DNA) 3′ Linker Ligation
RNA+SPIKE+adapter—heat at 70 C for 2 min→ice (better—20° C. ice)

Make ligation reaction mix consisting of:

| Ligation Mix | 1 tube | |
|---|---|---|
| Dephosphorylated RNA | 6 (6.5/7) | 70 C. |
| RNA adapter RiL-19 (40 μM) | 0.5 | 2 min → |
| 20 pmoles | | cold ice |
| Make master-mix in the following order: | | |
| 10× NEB ligase 1 Buffer | 2 μl | |
| DMSO (100%) | 1.8 μl | |
| ATP (100 mM) (from −80° C.) | 0.2 μl | |
| PEG 8000 (50%) | 8 μl | |
| RNase inhibitor | 0.3 μl | |
| T4 RNA Ligase 1, HiC, | 1.3 μl | 13.6 |
| 36 Units | | |
| Total | 20 μl | |

NOTE: use 1-10 pmoles of RiL-19 RNA adapter for 50-500 pg of RNA or 5-20 pmoles of RiL-19 RNA adapter for 1-10 ng of RNA.
T4 RNA Ligase 1, HiConc—custom NEB order, 30 U/μl
Mix well MANY times, shake well IN HANDS!! (shake every 10-40 min)
Incubate at 23 C for 1 hour 15 minutes.

B. Silane Linker cleanup Take 15 μl of Silane beads/sample, add some RLT, remove all super from the beads. Bind with 3.0× of freshly added RLT and 0.58× (RNA+RLT volume) EtOH, mix well.

| RNA ligation rxn | 20 μl |
|---|---|
| volume | |
| RLT with beads | 61 μl |
| mix well for 1 min | |
| EtOH | 65 μl |

mix well for 2-9 min

NOTE: (use 55-81 μl of EtOH for RNA of 150-50 nt AND FOR DEPLETION PROTOCOL and 35-50 μl of EtOH for RNA longer than 150 nt (150-500 nt long RNA) or NO DEPLETION). Use roughly 10 μl of Silane beads, if you used low RNA amount and 1-5 pmoles of RiL19 RNA adapter.

Wash beads in 123 ul of 70% EtOH twice out of magnet,

Mag Sep—discard S/N

Let air-dry at RT for 3-10 minutes (for first of 2 can skip air dry)

Elute 14.3 μl H₂O (no depletion protocol) or 15-24 μl H₂O (rRNAs depletion protocol)

C. First Strand cDNA
Take 14 μl of RNA+0.5 μl of AR17 short RT primer (20 μM stock) (10 pmoles)
Mix well—Heat the mixture to 70 C for 2 min and immediately place on cold ice(better −20° C. ice),
Add (in order on +0° C. ice):

| AffinityScript RT Mix | 1 Reaction |
|---|---|
| 10× RT Buffer | 2 μl |
| 100 mM DTT | 2 μl |
| 25 mM dNTP Mix | 0.8 μl |
| RNAse inhibitor | 0.4 μl |
| AffinityScript RT Enzyme | 0.8 μl |
| Total | 20 μl |

Close stripes, quickly, shake in hands well, spin 5 sec—put in HOT (54-55° C.) incubator (from ice to 55° C.). Incubate at 55° C. for 5 min and 54° C. for 50 min, 4° C.—1 min in PCR machine.
Do not kill RT enzyme with any heat, keep RNA/DNA hybrids intact.

D. Primer removal: Add 3 μl of ExoSap-it into 20 μl of RT reaction and shake at 37° C. for 12 min

E. Remove biotin labeled probes.
Take 20 ul streptavidin beads per sample, and wash it with 10 mM Tris pH7.5, 250 mM (200) LiCl, 20 mM EDTA, 0.1% Triton.
Add 1/9 volume (2.5 ul) of 10× concentrated of above wash buffer to the samples, than mix the samples with the beads.
Incubate at 60° C. for 15 min with beads, shaking
Elute and keep the samples.

F. RNA degradation after RT
Add 1M NaOH to each samples to 100 mM concentration (2.55 ul).
Incubate/shake in a Thermomixer at 70 C for 10 minutes.
Put it on ice, and neautralize with 2.55 ul 1M Acetic acid.

C. RT primer cleanup Use 12 μl of Silane beads/sample, add some RLT to the beads, remove all super from the beads. Bind with 3.0× of fresh RLT (with beads) and 0.6× (RNA+RLT volume) EtOH (see table below), mix well.

| Reaction mix | 25 μl |
|---|---|
| RLT with beads | 75 μl |
| mix well for 1 min | |
| EtOH | 65 μl |

mix well for 2-9 min

NOTE: use 50-65 μl of EtOH for RNA of 40-150 nt AND FOR DEPLETION or ExoSAPit protocols, especially for Protect-Seq

Wash beads in 123 ul of 70% EtOH twice out of magnet

Mag Sep—discard S/N

Let air-dry at RT for 3-10 minutes (for first of 2 can skip air dry)

Keep Silane beads, add 5.5 μl of H₂O to the beads (Some water will evaporate).

H. Second LIGATION (ssDNA/ssDNA) 3′ Linker Ligation on the beads
cDNA+adapter—heat at 75° C. for 2 min→ice (+0° C. ice, DO NOT freeze).
Make ligation reaction mix consisting of:

| Ligation Mix | 1 tube, μL | |
|---|---|---|
| cDNA | 5 (5.5) μl | 75 C. |
| | | 2 min → |
| | | ice |
| 3Tr3 DNA adapter, 40 | 0.5 μl | |
| pmoles (80 μM stock) | | |
| Add master-mix | | |
| 10× NEB Buffer | 2 μl | |
| DMSO (100%) | 0.8 μl | |
| ATP (100 mM) | 0.2 μl | |
| PEG 8000 (50%) | 9.5 μl | |
| T4 RNA Ligase 1, HC, | 1.6 μl | 14.1 |
| 45 U | | |
| total | 20 μl | |

Mix well using ultra low-retention tips (USA Scientific)
Incubate at 23 C for 2-4 hours or overnight (mix pipetting every hour if can)

I. Silane Linker cleanup Take extra 5 μl of Silane beads/sample, rinse with some RLT, remove super. Bind with 3.0× of fresh RLT and 0.58× (RNA+RLT volume) EtOH, mix well.

| RNA | 20 μl |
|---|---|
| RLT with beads | 61 μl |
| mix well for 1 min | |
| EtOH | 55 μl |

mix well for 2-9 min

NOTE:(use 50-60 μl of EtOH for RNA of 50-150 nt and 35-40 μl of EtOH for RNA longer than 150 nt (150-500 nt long RNA)
Wash beads in 123 ul of 70% EtOH twice out of magnet
Mag Sep—discard S/N
Let air-dry at RT for 3-10 minutes (for first of 2 can skip air dry)
Elute in 27 μl H₂O (use 22-23 μL for PCR, keep rest on beads until you done)

J. PCR Enrichment Make a mix consisting of:

| PCR Mix | 1 Reaction |
|---|---|
| cDNA | 23 μl |
| Barcoded Primer mix (25 μM of each) | 2 μl |
| Phusion MasterMix | 25 μl |
| Total | 50 μl |

Run using PCR program:

| | | | |
|---|---|---|---|
| 98 C. | 30 | Sec | |
| 98 C. | 15 | sec | 4 cycles |
| 67 C. | 30 | sec | |
| 72 C. | 30 | sec | |
| 98 C. | 15 | sec | 10 (7) cycles |
| 72 C. | 30 | sec | |
| 72 C. | 120 | sec | |

+4° C.

Use 10-12 cycles TOTAL if use started from 1-10 ng of poly-A RNA (NO depletion protocol) Use 12-16 cycles if You used 5-50 ng of RNA and depleted rRNAs or you used 50-500 pg of poly-A RNA or RNA w/o depletion.

K. SPRI Library cleanup USE 60 μl of SPRI beads/sample.
Add SPRI beads, mix well slow pipetting 5-40 times.
Let's stay for 5-9 mins. Put on magnet, wait 2-5 minutes.
Remove CLEAR super.
Wash beads twice with 70% Ethanol.
Dry beads for 2-5 minutes, elute in 21 μl of H₂O.
Repeat SPRI Clean-up with 1.1× beads

Measure library concentrations with Qubit.

Load 1 μl on BioAnalyzer HSDNA chip.

Reagents list:

ALL buffers, additives (ATP, DTT, DMSO, dNTPs, etc) must be stored in small aliquots at −80° C. w/o lazy excuses. Non-perishable buffers could be store at −25-45° C. freezer. Keep enzymes at good deep freezer −20-30° C.

1× T4 RNA Ligase Reaction Buffer:

50 mM Tris-HCl

10 mM MgCl₂

1 mM Dithiothreitol

pH 7.5@25° C. NEB catalog #B0216S

T4 RNA ligase 1, 3× high concentration, 30 U/μL, custom order from NEB, ask from Breton Hornblower hornblower@eb.com

Breton Hornblower, Ph.D., North East Account Manager

New England Biolabs, 617 967 0603

RNAse inhibitor, Murine (NEB)

ATP, 100 mM, Roche xxx (SQM) (keep in single use 3-5 μL aliquots at −80° C.)

PEG8000, 50% in H₂O, Sigma 83271-100ML-F, PCode:101129041

DMSO HYBRI-MAX, in vials endotoxin free, D2650, Sigma, 5×5 mL

RT set:

10× RT Buffer from Agilent for AffinityScript

100 mM DTT from Agilent or from Affimetrix

dNTPs (25 mM each) from NEB or 100 mM (same 25 mM each) from Agilent

AffinityScript Multiple-Temperature Reverse Transriptase from Agilent (any RNAseH-minus RT could be OK)

RNAse inhibitor, Murine (NEB)

After RT treatments:

ExoSAP-it—Affymetrix

0.5M EDTA, NaOH, Acetic Acid—any ultra-clean.

### Example 13

### Results for RAP-RNA (Using RAP to Examine RNA-RNA Interactions)

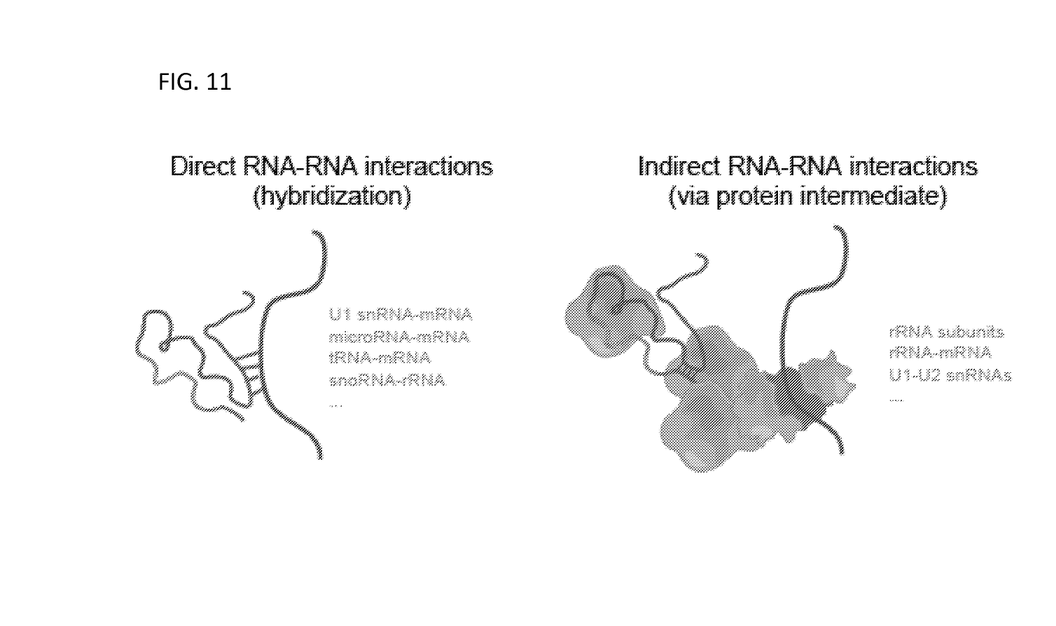

Mammalian genomes encode thousands of large and small noncoding RNAs (ncRNAs) many of which are implicated in diverse biological processes. While initial studies have begun to elucidate the cellular or organismal roles of ncRNAs, the molecular mechanisms by which they accomplish these functions remain largely uncharacterized. One strategy for characterizing these mechanisms is identification of other cellular components with which ncRNAs interact, such as specific proteins, DNA sites, and other RNAs. Notably, many canonical ncRNAs form intermolecular RNA complexes with other RNAs through either direct interactions mediated by base-pairing (e.g., microRNA-mRNA hybridization) or indirect interactions through protein intermediates (e.g., the multiple ncRNA components of the ribosome) (FIG. 11). In addition to these classic examples, numerous small nuclear RNAs (snRNAs) and large ncRNAs (lncRNAs) associate with protein complexes that regulate RNA splicing or transcription, suggesting they may target other RNAs as part of their regulatory function. These observation suggest that RNA-RNA interactions, along with RNA-DNA and RNA-protein interactions, represent a fundamental strategy used by many ncRNAs and that mapping these interactions may provide insight into ncRNA function and mechanism.

While investigations of RNA interactions have traditionally used in vitro affinity approaches, recent advances in RNA-centric biochemical purification techniques have highlighted the opportunity to systematically map RNA complexes in vivo. RNA Antisense Purification (RAP) has been used to study RNA localization to chromatin (RAP-DNA) and has provided new insight into RNA-DNA interactions. However, mapping intermolecular RNA-RNA interactions remains challenging. Recently, an initial study used proximity ligation to map RNA complexes bound to Ago2 [CLASH], providing a global view of miRNA-RNA interactions. However, this method has limited resolution for any given RNA and cannot distinguish between direct and indirect RNA-RNA interactions.

To address this challenge, RAP methods were developed to identify the intermolecular RNA-RNA interactions of a target RNA (RAP-RNA). RAP-RNA identifies endogenous RNA-RNA complexes through RNA capture with antisense oligos followed by high-throughput RNA sequencing. This approach provides a specific and systematic view of other RNAs that interact with a target RNA, and furthermore can distinguish between direct and indirect RNA-RNA interactions through use of crosslinking reagents with different reactivities with proteins and nucleic acids.

### Results

To identify direct and indirect RNA-RNA interactions, two related protocols, RAP-RNA and RAP-RNA-Direct, were developed. In the RAP-RNA-Direct protocol, direct RNA-RNA interactions were fixed in mouse embryonic stem (ES) cells with 4′-aminomethyltrioxalen (AMT), a psoralen crosslinker; when activated by long-wave ultraviolet (UV) light, psoralens generate inter-strand crosslinks between RNA uridine bases but do not react with proteins. In this approach, protein and DNA was eliminated prior to the purification, allowing specific identification of direct RNA-RNA interactions at high resolution. In the RAP-RNA protocol, a different crosslinking strategy was used to allow for the broadest capture of both direct and indirect RNA-RNA interactions: ES cells were fixed using formaldehyde and disuccinimydl glutarate (DSG), which together crosslink protein-RNA and protein-protein interactions and thus can capture both indirect interactions that occur via protein intermediates as well as direct interactions that are caged or flanked by proteins (e.g., U1 snRNA and associated proteins in complex with a pre-mRNA). In both protocols, target RNAs were purified with tiled biotinylated antisense oligos using a RAP protocol with improved sensitivity and specificity for low-abundance transcripts. Finally, co-purifying RNAs was sequenced using a custom strand-specific RNA-sequencing protocol.

RAP-RNA-Direct Captures U1 snRNA in Complex with 5′ Splice Sites

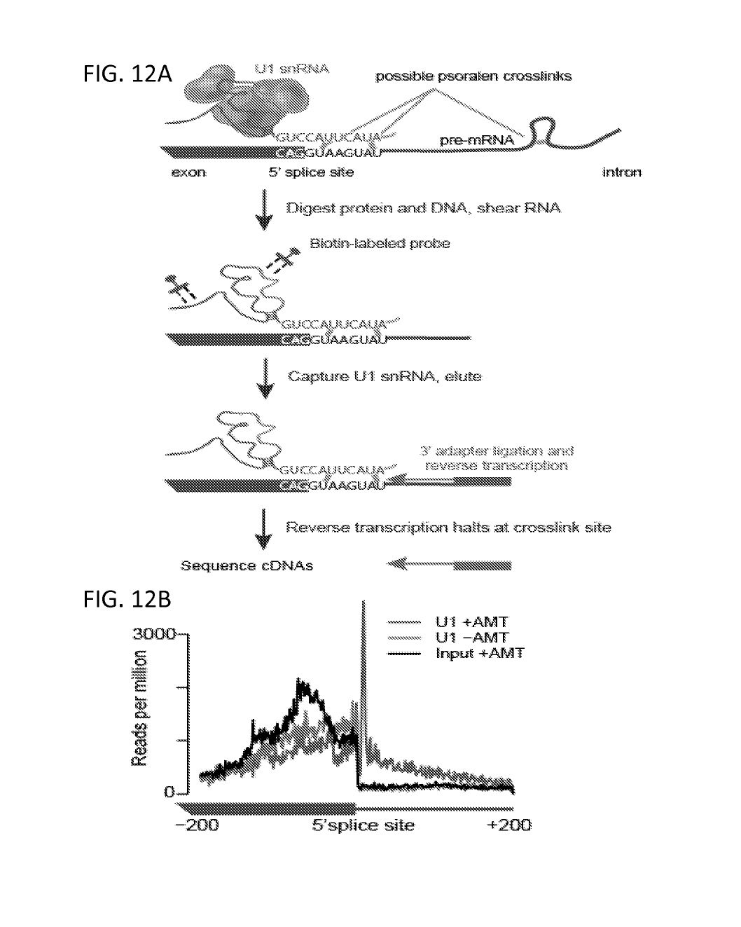

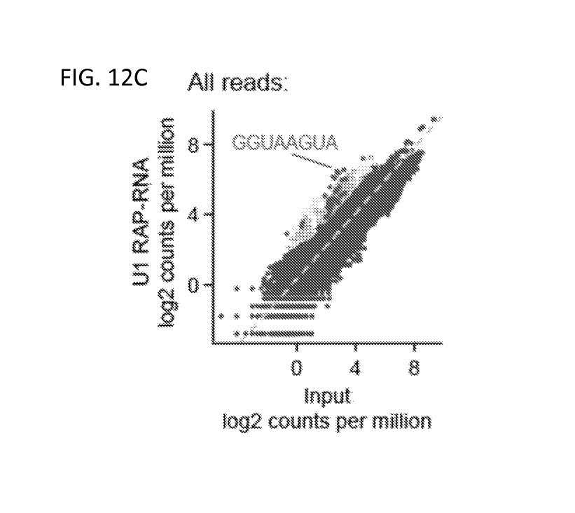

RAP-RNA-Direct was first developed to identify direct RNA-RNA interactions, which underpin many functional interactions for diverse regulatory RNAs including microRNAs, transfer RNAs, and snRNAs. To specifically identify hybridization-mediated interactions as opposed to indirect, protein-mediated interactions, live cells were treated with AMT and activated crosslinking through exposure to UV light (FIG. 12A). To eliminate the possibility of capturing indirectly associated RNAs, protein and DNA was digested before purifying the target RNA with antisense oligos. To map RNA-RNA interaction sites at high resolution, RNA was then chemically fragmented prior to capture and performed reverse transcription without reversal of crosslinks, leading to complementary DNA (cDNA) that terminates at or near the site of a crosslink (FIG. 12B).

To test this approach, the trial focused on U1 snRNA, which makes well-characterized base-pair contacts with pre-mRNAs at 5′ splice sites (we examined). U1 was captured using a set of three 50-nucleotide (nt) probes that span most of the 165-nt transcript but do not overlap with the sequence that base-pairs with pre-mRNAs. Upon capture of U1 using RAP-RNA-Direct, U1 comprised 90% of the sequencing reads, representing a 300-fold enrichment versus total input RNA. To test whether RAP had successfully captured the direct RNA interaction targets of U1, the density of sequencing read-ends was examined in the vicinity of 5′ splice sites. It was found that there was a sharp enrichment that peaked at eight bases downstream of the 5′ splice site (FIG. 12B), precisely where an AMT crosslink would occur in a canonical U1-pre-mRNA interaction (FIG. 12B). This enrichment extended for approximately 200 nucleotides into the intron (FIG. 12B), likely caused by intramolecular crosslinks in the pre-mRNA that blocked reverse transcription before it reached the 5′ splice site (FIG. 12A). This enrichment was present only in U1 RAP from crosslinked cells (+AMT) and not in input RNA or in U1 RAP from mock-crosslinked (−AMT) cells (FIG. 12B), demonstrating that this peak was not caused by direct probe-mediated capture or other purification artifacts.

While the analysis above focused on 5′ splice sites, the question was asked whether RAP-RNA-Direct could detect and recover the biology of U1-splice-site interactions de novo without prior knowledge about 5′ splice sites. To test this, 8-mers were counted upstream of each mappable sequencing read and it was found that the most enriched 8-mer motif exactly matched the consensus 5′ splice site sequence (17-fold enrichment, FIG. 12C). Most of the other significantly enriched motifs represented variants of the consensus (FIG. 12C). We note that because psoralens require opposing uridines to initiate a crosslink, the <5% of 5′ splice sites that deviate from the consensus motif and do not contain AU or UA dinucleotides are likely inaccessible when using a psoralen crosslinking agent. Nonetheless, the strong enrichment at 5′ splice sites and the de novo identification of the 5′ splice site motif shows that RAP-RNA-Direct can accurately and specifically detect direct interactions between the Ul snRNA and pre-mRNAs.

### Example 14

RNA Antisense Purification coupled with DNA sequencing (RAP-DNA) provides a method for purifying and mapping the in vivo DNA targets of large noncoding RNAs (lncRNAs). The following protocol outlines the experiments performed to purify the Xist noncoding RNA. Some modifications will likely be necessary for adaptation for other lncRNAs. 4.1 Lysate Preparation and Pre-clearing

1 Materials

1.1 Equipment

Sonication instrument (e.g., Branson Sonifier with microtip)

RT-PCR instrument (e.g., Roche LightCycler 480)

Magnetic rack for 1.5 mL tubes (e.g., Invitrogen DynaMag-2)

PCR machine

Microcentrifuge

NanoDrop spectrophotometer

Table-top centrifuge

Glass dounce

Liquid nitrogen

Agilent Bioanalyzer

1.2 Solutions

Cell Lysis Buffer

10 mM HEPES pH 7.5

20 mM KCl

1.5 mM MgCl2

0.5 mM EDTA

1 mM TCEP (add fresh)

0.5 mM PMSF (add fresh)

Nuclear Lysis Buffer

20 mM HEPES pH 7.5

50 mM KCl

1.5 mM MnCl2

1% NP-40

0.4% sodium deoxycholate

0.1% N-lauroylsarcosine

1 mM TCEP (add fresh)

0.5 mM PMSF (add fresh)

100× DNase Cofactor Solution

250 mM MnCl2

50 mM CaCl2

25× DNase Stop Solution

250 mM EDTA

125 mM EGTA

Hybridization Buffer (1×)

20 mM Tris-HCl pH 7.5

7 mM EDTA

3 mM EGTA

150 mM LiCl

1% NP-40

0.2% N-lauroylsarcosine

0.1% sodium deoxycyolate

3 M guanidine thiocyanate

2.5 mM TCEP

Wash Buffer

20 mM Tris-HCl pH 7.5

10 mM EDTA

1% NP-40

0.2% N-lauroylsarcosine

0.1% sodium deoxycyolate

3 M guanidine thiocyanate

2.5 mM TCEP

NLS Elution Buffer

20 mM Tris-HCl pH 7.5

10 mM EDTA

2% N-lauroylsarcosine

2.5 mM TCEP

1.3 Additional Materials and Reagents

Oligonucleotide library

Phusion High-Fidelity PCR Master Mix (NEB)

T7 RNA Polymerase and Buffer (NEB)

RNase Inhibitor (NEB)

100 mM ATP, CTP, GTP, UTP

10 mM Biotin-16-UTP (Roche)

Disuccinimidyl Glutarate (Pierce)

16% Formaldehyde Solution (Pierce)

2.5 M Glycine Solution

1 Å˜Phosphate Buffered Saline pH 7.5

BSA Fraction V Solution 7.5% (Invitrogen)

PCR Purification Kit (Qiagen)

RNeasy Mini Kit (Qiagen)

Buffer RLT (Qiagen)

TURBO DNase, 2U/μl (Invitrogen)

MyONE Streptavidin C1 Magnetic Beads (Invitrogen)

MyONE SILANE Magnetic Beads (Invitrogen)

Bioanalyzer: RNA 6000 Pico Kit, Small RNA Kit, High-Sensitivity DNA Kit (Agilent)

Agarose gel

PCR Primers

At several points, SILANE beads are used to clean up samples in place of column purifications or chloroform extractions. Use this protocol for a SILANE clean-up:

1. Aliquot out an appropriate volume of MyONE SILANE magnetic beads for the cleanup (capacity is at least 100 ng RNA or DNA per 10 μl of SILANE beads).

2. Rinse beads once in Buffer RLT (Qiagen).

3. Resuspend beads in 3.5 Å˜sample volume Buffer RLT (Qiagen) and add to sample.

4. Add 4.5 Å˜original sample volume isopropanol and mix well by pipet.

5. Incubate at room temperature for 2 minutes.

6. Place tube on magnetic rack. Wait 1-2 minutes for beads to separate, then remove and discard supernatant.

7. Wash by removing tube from magnetic rack, resuspending beads in 70% ethanol, capturing beads, and removing supernatant. Repeat wash step for a total of two washes.

8. Carefully remove all remaining 70% ethanol. Dry beads on the magnetic rack for 4-6 minutes or until dry (exact timing depends on bead volume).

9. Elute by resuspending beads in desired volume of H2O, then magnetically separating

and transferring eluate to a new tube.

2 Probe Generation

Goal: Generate long antisense biotinylated RNA probes that tile across the length of the target RNA. First, design and synthesize a library of 120-mer ssDNA oligos targeting a gene (or genes of interest). Next, amplify out a specific subset of probes targeting one of your genes of interest, and add a T7 promoter sequence to prepare for in vitro transcription. Finally, in vitro transcribe the T7 DNA template in the presence of biotinylated UTP to generate biotin-labeled probes. The method described below uses oligonucleotide library synthesis strategies to simultaneously generate pools of oligos targeting many genes of interest. An alternative strategy would be to use in vitro transcription to generate full-length biotinylated antisense RNA followed by controlled fragmentation (e.g., by heating in the presence of 10 mM ZnCl at 72° C. for approximately 10 minutes) to generate ˜60-180-mer RNA probes targeting a single gene of interest.

2.1 Oligo Design and Synthesis

1. Design 120-nucleotide oligos tiling every 15 nucleotides across the target RNA sequence. To avoid off-target hybridization, use BLAST to remove sequences that contain a perfect 30 base-pair match or an imperfect (90% identity) 60 base-pair match with another transcript or genomic region. Compare the oligos to RepeatMasker annotations and remove probes that contain more than 30 bases that overlap with a repeat annotation.

2. Design unique PCR tags (20 base-pairs) with a 60° C. annealing temperature that will not cross-hybridize with probe sequences or other genomic sequences. Design a unique pair of PCR tag for each gene of interest, and append these tags to all of the oligos targeting a single gene. The total length of each oligo will be 160 base pairs.

3. Order the pool of ssDNA oligos from an oligo library synthesis company (such as Agilent Technologies or CustomArray, Inc.). To improve synthesis of difficult sequences (such as probes containing poly-T sequences), synthesize both the desired ssDNA oligo as well as its reverse complement in the same pool.

4. Resuspend the lyophilized oligo pool in water to a final concentration of 10 nM.

2.2 T7 DNA Template Generation

5. Set up PCR reaction on ice:

| | |
|---|---|
| Oligo Pool (10 nM) | 1 μl |
| Tag Primer Left (25 μM) | 1 μl |
| Tag Primer Right (25 μM) | 1 μl |
| Phusion High-Fidelity 2x Master Mix (NEB) | 25 μl |
| H₂O | 23 μl |
| Total | 50 μl |

6. Run PCR program to enrich the desired subset of oligos from the pool:

| | | | |
|---|---|---|---|
| Initiation Denaturation | 98° C. | 30 seconds | 1 cycle |
| Denaturation | 98° C. | 10 seconds | |
| Annealing | 60° C. | 20 seconds | 25 cycles |
| Extension | 72° C. | 20 seconds | |
| Final Extension | 72° C. | 60 seconds | 1 cycle |
| Hold | 4° C. | hold | |

7. Clean up the PCR product using the Qiagen PCR Purification Kit according to manufacturer's protocol. Measure DNA yield using a NanoDrop spectrophotometer. Examine the dsDNA product on an agarose gel. If necessary, repeat Step 2 varying the annealing temperature or number of cycles until a single clean band is produced.

8. Perform a second round of amplification to add the T7 promoter sequence. Set up two PCR reactions to add the T7 promoter sequence onto one or the other end of the dsDNA template, allowing for generation of both antisense and sense probes during the in vitro transcription step.

| Diluted enriched dsDNA (~1 nM) | | | 1 μl |
|---|---|---|---|
| T7 Primer (25 μM) | | | 1 μl |
| Opposite Primer (25 μM) | | | 1 μl |
| Phusion High-Fidelity 2Å~Master Mix (NEB) | | | 25 μl |
| H₂O | | | 23 μl |
| Total | | | 50 μl |
| Initiation Denaturation | 98° C. | 30 seconds | 1 cycle |
| Denaturation | 98° C. | 10 seconds | |
| Annealing | 60° C. | 20 seconds | 3 cycles |
| Extension | 72° C. | 20 seconds | |
| Denaturation | 98° C. | 10 seconds | |
| Annealing | 68° C. | 20 seconds | 11 cycles |
| Extension | 72° C. | 20 seconds | |
| Final Extension | 72° C. | 60 seconds | 1 cycle |
| Hold | 4° C. | hold | |

9. Clean the PCR product using the Qiagen PCR Purification Kit according to manufacturer's protocol. Examine the dsDNA product on an agarose gel. Assess DNA yield using a NanoDrop spectrophotometer. If necessary, repeat Step 2 varying the annealing temperature or number of cycles until a single clean band is produced. If necessary, perform multiple PCR reactions to generate >200 ng of dsDNA T7 template for in vitro transcription.

2.3 In vitro Transcription of Biotinylated RNA

10. Set up the in vitro transcription reaction:

| | |
|---|---|
| T7 DNA template (~250 ng from Section 1.2) | 8 μl |
| 10Å~T7 RNA Pol Reaction Buffer (NEB) | 2 μl |
| 100 mM ATP | 1 μl |
| 100 mM CTP | 1 μl |
| 100 mM GTP | 1 μl |
| 100 mM UTP | 0.75 μl |
| 10 mM Biotin-16-UTP (Roche) | 2.5 μl |
| T7 RNA Polymerase (NEB) | 1.5 μl |
| 10 mM DTT | 2 μl |
| RNase Inhibitor (NEB) | 0.25 μl |
| Total | 20 μl |

11. Mix well by pipetting and incubate at 37° C. for >4 hours or overnight.

12. Denature RNA/DNA hybrids by incubating at 85° C. for 3 minutes.

13. Place immediately on ice for 1 minute.

14. Add 23 μl H₂O, 5 μl 10 Å˜TURBO DNase Buffer (Invitrogen), and 2 μl TURBO DNase (50 μl total volume). Note: TURBO DNase outperforms DNase I at high salt concentrations.

15. Incubate at 37° C. for 15 minutes to digest T7 DNA template.

16. Clean up product using Qiagen RNeasy Mini Kit according to manufacturer's protocol, except for adding 3.5 Å˜sample volume Buffer RLT and 4.5 Å˜sample volume 100% ethanol during the initial precipitation step. Elute in 50 μl H₂O.

17. Measure yield using a fluorometric assay. Expect 20-80 μg of final RNA product.

18. Denature RNA and run products on a gel or Small RNA Bioanalyzer Assay (Agilent) to visualize sizes. Expect a thick band around 160 nucleotides. The final RNA probe product will include the 120 nucleotides of target sequence as well as the 20 nucleotides of PCR tag on each end.

19. Store at −80° C. until use.

3 Lysate Preparation

Crosslink cells to fix in vivo lncRNA-chromatin interactions using a combination of formaldehyde and disuccinimidyl glutarate (DSG). Lyse cells to generate small DNA fragments while keeping RNA intact.

3.1 Cell Harvesting and Crosslinking

Note: This protocol describes the steps for adherent cells.

1. Grow adherent cells on 15-cm plates.

2. Resuspend 50-mg of DSG in 306 μl DMSO to create a 0.5 M stock solution of DSG.

3. Dilute DSG to 2 mM in PBS (prepare 10 mL for each 15-cm plate).

4. Remove media from cells by aspiration. Wash cells in plate with 10 mL room temperature PBS and remove by aspiration.

5. Add 10 mL of 2 mM DSG solution at room temperature.

6. Rock gently at room temperature for 45 minutes to crosslink.

7. Immediately before using, prepare a 3% formaldehyde solution in PBS preheated to 37° C. Use a fresh ampule of 16% formaldehyde (Pierce).

8. Remove DSG solution from cells and wash once with room temperature PBS.

9. Add 10 mL warmed 3% formaldehyde solution to cells. Incubate at 37° C. for 10 minutes, gently swirling by hand every 3 minutes.

10. Halt formaldehyde crosslinking by adding glycine to a final concentration of 100 mM. Incubate at 37° C. for 5 minutes.

11. Discard formaldehyde waste in appropriate disposal container.

12. Carefully wash three times with cold PBS, gently rocking plate for 1-2 minutes for each wash.

13. After last wash, add 2 mL of Scraping Buffer (ice cold PBS+0.5% BSA Fraction V) to each 15-cm plate. From here onward, keep cells at 4° C.

14. Scrape cells from plate and transfer to a 15-mL Falcon tube.

15. Centrifuge at 1000 Å˜g at 4° C. for 5 minutes to pellet cells.

16. Discard supernatant and resuspend pellet in 1 mL cold Scraping Buffer to break up the pellet. Add Scraping Buffer until cell concentration is ˜10 million cells per 1 mL.

17. Aliquot cells into microcentrifuge tubes (10 million cells each) and spin at 2000 Å˜g at 4° C. for 5 minutes.

18. Remove supernatant and flash freeze pellet in liquid nitrogen. Store until cell lysis at −80° C.

3.2 Cell Lysis

Note: All steps and buffers should be cooled to 4° C. unless otherwise stated.

19. Thaw cell pellets by completely resuspending 10 million cells in 1 mL Hypotonic Cell Lysis Buffer (add TCEP and PMSF fresh) in a 1.5 mL microcentrifuge tube.

20. Pellet cells by spinning at 3300 Å˜g for 7 minutes. Remove supernatant.

21. Gently resuspend swelled cells in 1 mL ice cold Cell Lysis Buffer+0.1% NP-40. Incubate on ice for 10 minutes.

22. Transfer to an ice-cold glass dounce of appropriate size (e.g., 2 mL). Homogenize cell lysate by douncing 20 Å˜.

23. Transfer cells back to a centrifuge tube and pellet nuclei by spinning at 3300 Å˜g for 7 minutes. Remove supernatant.

24. Resuspend nuclei in 540 μl of Nuclear Lysis Buffer (add TCEP, PMSF, RNase Inhibitor fresh). Incubate on ice for 10 minutes.

25. Sonicate using a Branson Sonifier fitted with a microtip using 5 Watts of power for 60 seconds in pulses (0.7 seconds on, 3.3 seconds off).

26. Add 6 ul 100 Å˜DNase Cofactor Solution and 100 μl TURBO DNase (Invitrogen).

27. Transfer to a 37° C. heat block and incubate at 37° C. for 10-12 minutes. Note: To optimize DNase timing and conditions, remove 5 μl aliquots every 5 minutes, quench with EDTA and EGTA on ice, and assay RNA and DNA sizes as described below.

28. Return sample to ice and immediately quench DNase reaction by adding 24 μl of 25 Å˜DNase Stop Solution. Mix immediately by pipetting.

29. Remove a 5 μl sample to check DNA and RNA sizes.

30. Mix the 600 μl of lysate with 1.5 mL of 1.4 Å˜concentrated Hybridization Buffer.

31. Clear lysate by spinning at maximum speed (16000 Å˜g) for 10 minutes.

32. Flash-freeze aliquots of lysate in liquid nitrogen and store at −80° C.

3.3 Check RNA and DNA Sizes

33. To check RNA and DNA sizes of saved aliquots of lysate (steps 28 and 30 above), add 2.5 μl Proteinase K, 2.5 μl of 5 M NaCl, and 15 μl of NLS Elution Buffer to 5 μl of saved lysate.

34. Incubate at 65° C. for 60 minutes.

35. Clean up sample using magnetic SILANE beads (see Reagents). Elute in 18 μl H₂O but do not remove from beads.

36. Split sample (with beads) in half (9 μl each) into RNA and DNA samples. To RNA sample, add 1 μl TURBO DNase Buffer and 1 μl TURBO DNase (Invitrogen). To RAP Protocol DNA sample, add 1 μl RNase Cocktail (Invitrogen). Incubate each sample at 37° C. for 10 minutes.

37. Repeat SILANE clean-up to purify DNA or RNA using the beads in the reaction. Elute in 10 μl H2O.

38. Assess fragment sizes with the Agilent Bioanalyzer (High-Sensitivity DNA for DNA and RNA Pico for RNA). The figures below show ideal sizes, although some variation is expected and may not significantly change the efficiency of the purification. DNA sizes after lysis (High-Sensitivity DNA Bioanalyzer) RNA sizes after lysis (RNA 6000 Pico Bioanalyzer Assay)

4 RAP

Purify genomic DNA and chromatin that is crosslinked to a target lncRNA of interest. Perform the purification in parallel with sense and antisense probes to control for direct probe-DNA hybridization and other potential artifacts.

4.1 Lysate Preparation and Pre-Clearing

1. Thaw lysate corresponding to 5 million cells for each sample (˜1 mL).

2. Aliquot out 100 μl MyONE Streptavidin C1 magnetic beads (Invitrogen) for each purification from 5 million cells.

3. Wash beads twice in 100 μl Hybridization Buffer, using a magnetic rack to capture beads. Remove and discard supernatant.

4. Resuspend beads in 100 μl Hybridization Buffer. Add beads to lysate.

5. Incubate at 45° C. for 20 minutes.

6. Magnetically separate and transfer supernatant (pre-cleared lysate) to a clean tube. Repeat this step to completely remove beads.

7. Save 2 μl of pre-cleared lysate on ice as RNA input and 18 μl as DNA input.

4.2 Hybridization, Capture, Wash

8. Aliquot out 100 ng of biotinylated RNA probe for each purification.

9. Denature probe at 85° C. for 3 minutes and then transfer immediately to ice.

10. Add probe to lysate and immediately transfer to a 45° C. heat block or hybridization oven.

11. Incubate at 45° C. for 2-3 hours.

12. Just before use, aliquot out 25 μl of Streptavidin C1 magnetic beads for each purification sample. Wash twice and resuspend in 25 μl of Hybridization Buffer.

13. Add streptavidin-coated beads to purification sample and incubate at 45° C. for 15 minutes, shaking at 1400 rpm on a thermal mixer.

14. Place sample on magnet. Wait 1 minute for complete magnetic separation, then remove and discard supernatant. Resuspend beads in 100 μl Wash Buffer, then incubate at 45° C. for 3-4 minutes.

15. Before removing final wash, resuspend beads and remove 10% volume of beads (10 μl) for RNA analysis. Transfer the remaining 90% volume of beads (90 μl) to a new tube for DNA analysis.

4.3 RNA Elution and Analysis

16. Magnetically separate and remove final wash.

17. Add 20 μl NLS Elution Buffer and incubate at 94° C. for 5 minutes to release biotin and reverse crosslinks. Magnetically separate and save eluate.

18. Add 2.5 μl 5M NaCl and 2.5 μl Proteinase K.

19. Dilute the saved input sample in NLS Elution Buffer, adding NaCl and Proteinase K.

20. Incubate at 65° C. for 60 minutes.

21. Clean up sample using ethanol precipitation onto SILANE beads (see Reagents). Add 12.5 μl H2O to elute but do not remove from beads.

22. Add 1.5 μl TURBO DNase Buffer and 1 μl DNase to digest DNA. Incubate at 37° C. for 10 minutes.

23. Repeat SILANE clean-up using beads already in sample. Elute in 12 μl H₂O

24. Reverse-transcribe captured RNA (as well as the RNA probe) using AffinityScript Reverse Transcriptase (Agilent) according to manufacturer's protocol (20 μl reaction).

25. Dilute the reverse transcription reactions with H₂O to 200 μl volume total.

26. Analyze captured RNA by qPCR to determine enrichment (by comparing to the input or sense-probe control samples) and yield (by comparing to the input sample). qPCR primers should be designed to have amplicons >160-bp to avoid exponential amplification of the cDNA created from the RNA probes. Even with this longamplicon primer design, the signal from the probe may mask the signal from the captured RNA; thus, to ensure that the qPCR signals do not reflect measurement of the RNA probe, it may prove helpful during optimization experiments to include a “probe-only” control purification in which RNA probe is incubated in hybridization buffer without lysate, then captured, washed, and eluted in parallel with the other samples.

4.4 DNA Elution and Analysis

27. Magnetically separate and remove final wash.

28. Add 65 μl NLS Elution Buffer, 8 μl 5 M NaCl, and 8 μl Proteinase K to each sample.

29. Dilute the saved input sample in NLS Elution Buffer, adding NaCl and Proteinase K.

30. Incubate at 60° C. overnight to completely reverse formaldehyde crosslinks.

31. Clean-up DNA samples with SILANE beads.

32. Assay DNA yields and enrichments using qPCR, or generate DNA sequencing libraries using the NEBNext ChIP-Seq Library Prep Master Mix Set for Illumina (NEB) as previously described [cite]. qPCR primers targeting introns or nearby genomic DNA (that does not overlap with the probe) should show strong enrichment (20-100 fold) in the antisense-probe purification versus the sense-probe control and versus input.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of this disclosure and these claims.

1. A method for isolating a target nucleic acid molecule of interest from a sample, comprising:
contacting a sample comprising a target nucleic acid molecule of interest with at least one single stranded nucleic acid targeting probe that specifically hybridizes to the nucleic acid molecule of interest under highly stringent conditions, wherein the targeting probe comprises a nucleic acid sequence from at least about 30 bases in length to about the length of the target nucleic acid molecule of interest, and wherein the nucleic acid targeting probe further comprises one or more capture moieties covalently linked the targeting probe and optionally a detectable label;
contacting the sample with protein-nucleic acid crosslinking agent, a nucleic acid-nucleic acid crosslinking agent, a protein-protein crosslinking agent or any combination thereof before contacting the sample with a targeting probe, thereby isolating any protein and/or nucleic acids crosslinked to the target nucleic molecule of interest; and
capturing the at least one targeting probe via the one or more capture moieties, thereby isolating the target nucleic acid molecule of interest specifically bound to the targeting probe.

2. The method of claim 1, further comprising removing from the sample, the isolated target nucleic acid molecule of interest specifically bound to the targeting probe, thereby depleting the sample of the target nucleic acid molecule of interest.

3. The method of claim 1, wherein contacting the sample with at least one single stranded nucleic acid targeting probe comprises contacting the sample with a plurality of single stranded nucleic acid targeting probes.

4. The method of claim 3 wherein the different single stranded nucleic acid targeting probes in the plurality are complementary to different nucleic acid sequences within the target nucleic molecule of interest.

5. The method of claim 4, wherein the different single stranded nucleic acid targeting probes in the plurality are selected to tile across the target nucleic molecule of interest and optionally the probes overlap when tiled across the target nucleic molecule of interest.

6. (canceled)

7. The method of claim 1, wherein the targeting probes, comprise one or more specific nucleic acid sequence tags, and optionally wherein the one or more specific nucleic acid sequence tags include a nucleic acid sequence tag for distinguishing between different target nucleic acids, a nucleic acid sequence tag for distinguishing between subregions of the target nucleic acid, a nucleic acid sequence tag for distinguishing a set of target nucleic acids from another set of nucleic acids, a PCR tag, and/or a nucleic acid sequence tag for excluding a specific subregion or subregions of the target nucleic acid.

8. (canceled)

9. (canceled)

10. (canceled)

11. (canceled)

12. The method of claim 1, wherein the targeting probes, comprise an RNA promotor and/or a series of hierarchical nucleic acid sequence tags.

13. (canceled)

14. (canceled)

15. (canceled)

16. (canceled)

17. (canceled)

18. The method of claim 1, further comprising washing the captured probes at a selected combination of temperature, denaturing condition, salt concentration, and/or other condition sufficient to substantially remove molecules that non-specifically bind to the targeting probe.

19. (canceled)

20. The method of claim 1, wherein the one or more capture moieties is captured on a solid support and/or with a capture moiety specific binding agent that specifically binds to the one or more capture moieties optionally attached to the solid support.

21. (canceled)

22. The method of claim 20, further comprising eluting the captured targeting probes.

23. The method of claim 22, wherein elution comprises elution with an agent that targets double stranded nucleic acid regions, such as a double stranded RNA region, contacting the captured probes with specific nucleases, chemicals, or other agents that specifically target double stranded nucleic acid regions, boiling the captured probes in a denaturant-containing solution, contacting the captured probes with a dsRNA-specific RNase, reverse crosslinking and contacting the probes with proteinase K, degrading RNA in high alkaline conditions, specific disruption of the interaction between the probe and the target RNA, specific destruction of the probe or target RNA and or specific disruption of the interaction between the interaction between the probe and the solid support.

24. (canceled)

25. (canceled)

26. (canceled)

27. (canceled)

28. (canceled)

29. (canceled)

30. (canceled)

31. (canceled)

32. (canceled)

33. The method of claim 1, wherein the one or more capture moieties comprises biotin and/or wherein the capture moiety specific binding agent comprises streptavidin.

34. (canceled)

35. The method of claim 1, wherein the targeting probe is an RNA probe, a DNA probe, a locked nucleic acid (LNA) probe, or a hybrid RNA/DNA probe and/or wherein the targeting probe comprises biotin-16-UTP.

36. (canceled)

37. The method of claim 1, wherein the target nucleic molecule of interest is RNA.

38. (canceled)

39. (canceled)

40. The method of claim 1, further comprising determining the identity of protein and/or nucleic acid crosslinked to the target nucleic acid.

41. The method of claim 40, wherein determining the identity of the protein comprises the use of an antibody and/or mass spectrometry.

42. (canceled)

43. The method of claim 40, wherein the determining the identity of the nucleic acid comprises sequencing and/or PCR.

44. The method of claim 40, further comprising correlating the identified identity of protein and/or nucleic acid crosslinked to the target nucleic acid to a disease or condition or an environmental condition.

45. (canceled)

46. (canceled)

47. (canceled)

48. (canceled)

49. (canceled)

50. (canceled)

51. A method for diagnosing a disease or condition, the method comprising:
identifying protein and/or nucleic acid crosslinked to the target nucleic acid according to claim 1 and comparing the identifying protein and/or nucleic acid crosslinked to the target nucleic acid with a control indicative of a disease or condition, wherein a similarity between the identified protein and/or nucleic acid crosslinked and the control diagnoses the disease or condition.

52. (canceled)

53. (canceled)

54. A method of identifying a modulator of protein and/or nucleic acid binding to a target nucleic acid, the method comprising:
identifying protein and/or nucleic acid crosslinked to the target nucleic acid according to claim 1, wherein the sample has been contacted with a test agent prior to crosslinking; and
comparing the identified protein and/or nucleic acid crosslinked to the target nucleic acid with a control, wherein a difference between the identified protein and/or nucleic acid crosslinked to the target nucleic acid and the control identifies the test agent as modulator of protein and/or nucleic acid binding to the target nucleic acid.

55. (canceled)

56. (canceled)

57. (canceled)

58. A set of targeting probes specific for one or more target nucleic molecules of interest, comprising a plurality of different single stranded nucleic acid probes have a nucleic acid sequence complementary to different nucleic acid sequences within the one or more target nucleic molecules of interest.

59. The set of targeting probes of claim 58, wherein the different single stranded nucleic acid targeting probes in the plurality are selected to tile, and optionally overlap, across the one or more target nucleic molecules of interest.

60. (canceled)

61. (canceled)

62. The set of targeting probes of claim 58, wherein the probes comprise one or more specific nucleic acid sequence tags.

63. The set of targeting probes of claim 62, wherein the one or more specific nucleic acid sequence tags include a nucleic acid sequence tag for distinguishing between different target nucleic acids, a nucleic acid sequence tag for distinguishing between subregions of the target nucleic acid, a nucleic acid sequence tag for distinguishing a set of target nucleic acids from another set of nucleic acids, PCR tags, and/or a nucleic acid sequence tag for excluding a specific subregion or subregions of the target nucleic acid.

64. (canceled)

65. (canceled)

66. (canceled)

67. The set of targeting probes of claim 58, wherein the targeting probes, comprise an RNA promotor and/or a series of hierarchical nucleic acid sequence tags.

68. (canceled)

69. (canceled)

70. (canceled)

71. (canceled)

72. (canceled)

73. A kit, comprising the set of targeting probes of claim 58.
